SEQUENCE LISTING (20 sequences)

SEQ ID NO: 1 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - DNA, 145 bp, Adeno-associated virus
  source 1..145: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 3 - DNA, 448 bp, Homo sapiens
  source 1..448: mol_type = genomic DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 4 - DNA, 276 bp, Gallus gallus
  source 1..276: mol_type = genomic DNA
tcgaggtgagccccacgttctgcttcactctccccatctcccccccctccccacccccaattttgtatttatttattttttaattattttatgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 5 - DNA, 198 bp, Simian virus 40
  source 1..198: mol_type = genomic DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttag

SEQ ID NO: 6 - skipped

SEQ ID NO: 7 - DNA, 304 bp, Cytomegalovirus
  source 1..304: mol_type = genomic DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 8 - DNA, 95 bp, Simian virus 40
  source 1..95: mol_type = genomic DNA
gctctaaggtaaatataaaatttttaagtgtataatgtgttaaactactgattctaattgtttctctcttttagattccaacctttggaactgat

SEQ ID NO: 9 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 10 - protein, 349 aa, Streptococcus equi
MKTIAYPNKPHSLSAGLLTAIAIFSLASSNITYADDYQRNATEAYAKEVPHQITSVWSKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 11 - DNA, 2211 bp, Adeno-associated virus
  source 1..2211: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 12 - DNA, 2880 bp, Homo sapiens
  source 1..2880: mol_type = genomic DNA
atgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccaggtcagatggatcctggttggcatgtgtcctctgtgaccaggagtgccacagagggcccttcctactctgaacagctgggtgccaaaagtgggccaaatgggcacccctataacagaacaaatcgctcacgaatgccaaatctgaatgatttaaaagagacagccttg

SEQ ID NO: 13 - DNA, 3090 bp, Homo sapiens
  source 1..3090: mol_type = genomic DNA
atgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccagacggtggatgtgatggcagaagacagagacaccattctggaccccaagatagacgcttcatgttaaggacgacagaacaacaaggagaatacttctgctgtggtgacccaaagaagcctcacactccgtgcgtcccaaaccgagcccttcatcgtccaatctccagtcctgctccctatccagtactccaggtccgaggcacttccatgtgcccgacactccaggtccgaggcactgatgctttcagctgcccaacccagcaatccgggttctctttcttcgtgagacacgttatgagggaagccctgattcacagggcccaggtaaaccaagctgcgctcctgacataccatgagaatgcggcactgacgggcaag

SEQ ID NO: 14 - DNA, 2880 bp, synthetic construct
  source 1..2880: mol_type = other DNA
atgaagattcctaatattgggaatgtgatgaataagtttgagattctgggggtggtgggggagggggcttatggggtggtgctgaagtgtaggcataaggagacacatgagattgtggctattaagaagtttaaggattctgaggagaatgaggaggtgaaggagacaacactgagggagctgaagatgctgaggacactgaagcaggagaatattgtggagctgaaggaggcttttaggaggagggggaagctgtatctggtgtttgagtatgtggagaagaatatgctggagctgctggaggagatgcctaatggggtgcctcctgagaaggtgaagtcttatatttatcagctgattaaggctattcattggtgtcataagaatgatattgtgcatagggatattaagcctgagaatctgctgatttctcataatgatgtgctgaagctgtgtgattttgggtttgctaggaatctgtctgaggggaataatgctaattatacagagtatgtggctacaaggtggtataggtctcctgagctgctgctgggggctccttatgggaagtctgtggatatgtggtctgtggggtgtattctgggggagctgtctgatgggcagcctctgtttcctggggagtctgagattgatcagctgtttacaattcagaaggtgctggggcctctgccttctgagcagatgaagctgttttattctaatcctaggtttcatgggctgaggtttcctgctgtgaatcatcctcagtctctggagaggaggtatctggggattctgaattctgtgctgctggatctgatgaagaatctgctgaagctggatcctgctgataggtatctgacagagcagtgtctgaatcatcctacatttcagacacagaggctgctggataggtctccttctaggtctgctaagaggaagccttatcatgtggagtcttctacactgtctaataggaatcaggctgggaagtctacagctctgcagtctcatcataggtctaattctaaggatattcagaatctgtctgtggggctgcctagggctgatgaggggctgcctgctaatgagtcttttctgaatgggaatctggctggggcttctctgtctcctctgcatacaaagacatatcaggcttcttctcagcctgggtctacatctaaggatctgacaaataataatattcctcatctgctgtctcctaaggaggctaagtctaagacagagtttgattttaatattgatcctaagccttctgaggggcctgggacaaagtatctgaagtctaattctaggtctcagcagaataggcattcttttatggagtcttctcagtctaaggctgggacactgcagcctaatgagaagcagtctaggcattcttatattgatacaattcctcagtcttctaggtctccttcttataggacaaaggctaagtctcatggggctctgtctgattctaagtctgtgtctaatctgtctgaggctagggctcagattgctgagccttctacatctaggtattttccttcttcttgtctggatctgaattctcctacatctcctacacctacaaggcattctgatacaaggacactgctgtctccttctgggaggaataataggaatgaggggacactggattctaggaggacaacaacaaggcattctaagacaatggaggagctgaagctgcctgagcatatggattcttctcattctcattctctgtctgctcctcatgagtctttttcttatgggctggggtatacatctcctttttcttctcagcagaggcctcataggcattctatgtatgtgacaagggataaggtgagggctaaggggctggatgggtctctgtctattgggcaggggatggctgctagggctaattctctgcagctgctgtctcctcagcctggggagcagctgcctcctgagatgacagtggctaggtcttctgtgaaggagacatctagggaggggacatcttcttttcatacaaggcagaagtctgaggggggggtgtatcatgatcctcattctgatgatgggacagctcctaaggagaataggcatctgtataatgatcctgtgcctaggagggtggggtctttttatagggtgccttctcctaggcctgataattcttttcatgagaataatgtgtctacaagggtgtcttctctgccttctgagtcttcttctgggacaaatcattctaagaggcagcctgcttttgatccttggaagtctcctgagaatatttctcattctgagcagctgaaggagaaggagaagcaggggttttttaggtctatgaagaagaagaagaagaagtctcagacagtgcctaattctgattctcctgatctgctgacactgcagaagtctattcattctgcttctacaccttcttctaggcctaaggagtggaggcctgagaagatttctgatctgcagacacagtctcagcctctgaagtctctgaggaagctgctgcatctgtcttctgcttctaatcatcctgcttcttctgatcctaggtttcagcctctgacagctcagcagacaaagaattctttttctgagattaggattcatcctctgtctcaggcttctggggggtcttctaatattaggcaggagcctgctcctaaggggaggcctgctctgcagctgcctgggcagatggatcctgggtggcatgtgtcttctgtgacaaggtctgctacagaggggccttcttattctgagcagctgggggctaagtctgggcctaatgggcatccttataataggacaaataggtctaggatgcctaatctgaatgatctgaaggagacagctctg

SEQ ID NO: 15 - DNA, 2880 bp, synthetic construct
  source 1..2880: mol_type = other DNA
atgaagattccaaatattgggaatgtgatgaataagtttgagattctgggggtggtgggggagggggcatatggggtggtgctgaagtgtaggcataaggagacacatgagattgtggcaattaagaagtttaaggattcagaggagaatgaggaggtgaaggagacaacactgagggagctgaagatgctgaggacactgaagcaggagaatattgtggagctgaaggaggcatttaggaggagggggaagctgtatctggtgtttgagtatgtggagaagaatatgctggagctgctggaggagatgccaaatggggtgccaccagagaaggtgaagtcatatatttatcagctgattaaggcaattcattggtgtcataagaatgatattgtgcatagggatattaagccagagaatctgctgatttcacataatgatgtgctgaagctgtgtgattttgggtttgcaaggaatctgtcagaggggaataatgcaaattatacagagtatgtggcaacaaggtggtataggtcaccagagctgctgctgggggcaccatatgggaagtcagtggatatgtggtcagtggggtgtattctgggggagctgtcagatgggcagccactgtttccaggggagtcagagattgatcagctgtttacaattcagaaggtgctggggccactgccatcagagcagatgaagctgttttattcaaatccaaggtttcatgggctgaggtttccagcagtgaatcatccacagtcactggagaggaggtatctggggattctgaattcagtgctgctggatctgatgaagaatctgctgaagctggatccagcagataggtatctgacagagcagtgtctgaatcatccaacatttcagacacagaggctgctggataggtcaccatcaaggtcagcaaagaggaagccatatcatgtggagtcatcaacactgtcaaataggaatcaggcagggaagtcaacagcactgcagtcacatcataggtcaaattcaaaggatattcagaatctgtcagtggggctgccaagggcagatgaggggctgccagcaaatgagtcatttctgaatgggaatctggcaggggcatcactgtcaccactgcatacaaagacatatcaggcatcatcacagccagggtcaacatcaaaggatctgacaaataataatattccacatctgctgtcaccaaaggaggcaaagtcaaagacagagtttgattttaatattgatccaaagccatcagaggggccagggacaaagtatctgaagtcaaattcaaggtcacagcagaataggcattcatttatggagtcatcacagtcaaaggcagggacactgcagccaaatgagaagcagtcaaggcattcatatattgatacaattccacagtcatcaaggtcaccatcatataggacaaaggcaaagtcacatggggcactgtcagattcaaagtcagtgtcaaatctgtcagaggcaagggcacagattgcagagccatcaacatcaaggtattttccatcatcatgtctggatctgaattcaccaacatcaccaacaccaacaaggcattcagatacaaggacactgctgtcaccatcagggaggaataataggaatgaggggacactggattcaaggaggacaacaacaaggcattcaaagacaatggaggagctgaagctgccagagcatatggattcatcacattcacattcactgtcagcaccacatgagtcattttcatatgggctggggtatacatcaccattttcatcacagcagaggccacataggcattcaatgtatgtgacaagggataaggtgagggcaaaggggctggatgggtcactgtcaattgggcaggggatggcagcaagggcaaattcactgcagctgctgtcaccacagccaggggagcagctgccaccagagatgacagtggcaaggtcatcagtgaaggagacatcaagggaggggacatcatcatttcatacaaggcagaagtcagaggggggggtgtatcatgatccacattcagatgatgggacagcaccaaaggagaataggcatctgtataatgatccagtgccaaggagggtggggtcattttatagggtgccatcaccaaggccagataattcatttcatgagaataatgtgtcaacaagggtgtcatcactgccatcagagtcatcatcagggacaaatcattcaaagaggcagccagcatttgatccatggaagtcaccagagaatatttcacattcagagcagctgaaggagaaggagaagcaggggttttttaggtcaatgaagaagaagaagaagaagtcacagacagtgccaaattcagattcaccagatctgctgacactgcagaagtcaattcattcagcatcaacaccatcatcaaggccaaaggagtggaggccagagaagatttcagatctgcagacacagtcacagccactgaagtcactgaggaagctgctgcatctgtcatcagcatcaaatcatccagcatcatcagatccaaggtttcagccactgacagcacagcagacaaagaattcattttcagagattaggattcatccactgtcacaggcatcaggggggtcatcaaatattaggcaggagccagcaccaaaggggaggccagcactgcagctgccagggcagatggatccagggtggcatgtgtcatcagtgacaaggtcagcaacagaggggccatcatattcagagcagctgggggcaaagtcagggccaaatgggcatccatataataggacaaataggtcaaggatgccaaatctgaatgatctgaaggagacagcactg

SEQ ID NO: 16 - DNA, 2880 bp, synthetic construct
  source 1..2880: mol_type = other DNA
atgaagataccaaatataggtaatgtaatgaataagtttgaaatactaggtgtagtaggtgaaggtgcatatggtgtagtactaaagtgtaggcataaggaaacacatgaaatagtagcaataaagaagtttaaggattcagaagaaaatgaagaagtaaaggaaacaacactaagggaactaaagatgctaaggacactaaagcaagaaaatatagtagaactaaaggaagcatttaggaggaggggtaagctatatctagtatttgaatatgtagaaaagaatatgctagaactactagaagaaatgccaaatggtgtaccaccagaaaaggtaaagtcatatatatatcaactaataaaggcaatacattggtgtcataagaatgatatagtacatagggatataaagccagaaaatctactaatatcacataatgatgtactaaagctatgtgattttggttttgcaaggaatctatcagaaggtaataatgcaaattatacagaatatgtagcaacaaggtggtataggtcaccagaactactactaggtgcaccatatggtaagtcagtagatatgtggtcagtaggttgtatactaggtgaactatcagatggtcaaccactatttccaggtgaatcagaaatagatcaactatttacaatacaaaaggtactaggtccactaccatcagaacaaatgaagctattttattcaaatccaaggtttcatggtctaaggtttccagcagtaaatcatccacaatcactagaaaggaggtatctaggtatactaaattcagtactactagatctaatgaagaatctactaaagctagatccagcagataggtatctaacagaacaatgtctaaatcatccaacatttcaaacacaaaggctactagataggtcaccatcaaggtcagcaaagaggaagccatatcatgtagaatcatcaacactatcaaataggaatcaagcaggtaagtcaacagcactacaatcacatcataggtcaaattcaaaggatatacaaaatctatcagtaggtctaccaagggcagatgaaggtctaccagcaaatgaatcatttctaaatggtaatctagcaggtgcatcactatcaccactacatacaaagacatatcaagcatcatcacaaccaggttcaacatcaaaggatctaacaaataataatataccacatctactatcaccaaaggaagcaaagtcaaagacagaatttgattttaatatagatccaaagccatcagaaggtccaggtacaaagtatctaaagtcaaattcaaggtcacaacaaaataggcattcatttatggaatcatcacaatcaaaggcaggtacactacaaccaaatgaaaagcaatcaaggcattcatatatagatacaataccacaatcatcaaggtcaccatcatataggacaaaggcaaagtcacatggtgcactatcagattcaaagtcagtatcaaatctatcagaagcaagggcacaaatagcagaaccatcaacatcaaggtattttccatcatcatgtctagatctaaattcaccaacatcaccaacaccaacaaggcattcagatacaaggacactactatcaccatcaggtaggaataataggaatgaaggtacactagattcaaggaggacaacaacaaggcattcaaagacaatggaagaactaaagctaccagaacatatggattcatcacattcacattcactatcagcaccacatgaatcattttcatatggtctaggttatacatcaccattttcatcacaacaaaggccacataggcattcaatgtatgtaacaagggataaggtaagggcaaagggtctagatggttcactatcaataggtcaaggtatggcagcaagggcaaattcactacaactactatcaccacaaccaggtgaacaactaccaccagaaatgacagtagcaaggtcatcagtaaaggaaacatcaagggaaggtacatcatcatttcatacaaggcaaaagtcagaaggtggtgtatatcatgatccacattcagatgatggtacagcaccaaaggaaaataggcatctatataatgatccagtaccaaggagggtaggttcattttatagggtaccatcaccaaggccagataattcatttcatgaaaataatgtatcaacaagggtatcatcactaccatcagaatcatcatcaggtacaaatcattcaaagaggcaaccagcatttgatccatggaagtcaccagaaaatatatcacattcagaacaactaaaggaaaaggaaaagcaaggtttttttaggtcaatgaagaagaagaagaagaagtcacaaacagtaccaaattcagattcaccagatctactaacactacaaaagtcaatacattcagcatcaacaccatcatcaaggccaaaggaatggaggccagaaaagatatcagatctacaaacacaatcacaaccactaaagtcactaaggaagctactacatctatcatcagcatcaaatcatccagcatcatcagatccaaggtttcaaccactaacagcacaacaaacaaagaattcattttcagaaataaggatacatccactatcacaagcatcaggtggttcatcaaatataaggcaagaaccagcaccaaagggtaggccagcactacaactaccaggtcaaatggatccaggttggcatgtatcatcagtaacaaggtcagcaacagaaggtccatcatattcagaacaactaggtgcaaagtcaggtccaaatggtcatccatataataggacaaataggtcaaggatgccaaatctaaatgatctaaaggaaacagcacta

SEQ ID NO: 17 - DNA, 3090 bp, synthetic construct
  source 1..3090: mol_type = other DNA
atgaagattcctaatattgggaatgtgatgaataagtttgagattctgggggtggtgggggagggggcttatggggtggtgctgaagtgtaggcataaggagacacatgagattgtggctattaagaagtttaaggattctgaggagaatgaggaggtgaaggagacaacactgagggagctgaagatgctgaggacactgaagcaggagaatattgtggagctgaaggaggcttttaggaggagggggaagctgtatctggtgtttgagtatgtggagaagaatatgctggagctgctggaggagatgcctaatggggtgcctcctgagaaggtgaagtcttatatttatcagctgattaaggctattcattggtgtcataagaatgatattgtgcatagggatattaagcctgagaatctgctgatttctcataatgatgtgctgaagctgtgtgattttgggtttgctaggaatctgtctgaggggaataatgctaattatacagagtatgtggctacaaggtggtataggtctcctgagctgctgctgggggctccttatgggaagtctgtggatatgtggtctgtggggtgtattctgggggagctgtctgatgggcagcctctgtttcctggggagtctgagattgatcagctgtttacaattcagaaggtgctggggcctctgccttctgagcagatgaagctgttttattctaatcctaggtttcatgggctgaggtttcctgctgtgaatcatcctcagtctctggagaggaggtatctggggattctgaattctgtgctgctggatctgatgaagaatctgctgaagctggatcctgctgataggtatctgacagagcagtgtctgaatcatcctacatttcagacacagaggctgctggataggtctccttctaggtctgctaagaggaagccttatcatgtggagtcttctacactgtctaataggaatcaggctgggaagtctacagctctgcagtctcatcataggtctaattctaaggatattcagaatctgtctgtggggctgcctagggctgatgaggggctgcctgctaatgagtcttttctgaatgggaatctggctggggcttctctgtctcctctgcatacaaagacatatcaggcttcttctcagcctgggtctacatctaaggatctgacaaataataatattcctcatctgctgtctcctaaggaggctaagtctaagacagagtttgattttaatattgatcctaagccttctgaggggcctgggacaaagtatctgaagtctaattctaggtctcagcagaataggcattcttttatggagtcttctcagtctaaggctgggacactgcagcctaatgagaagcagtctaggcattcttatattgatacaattcctcagtcttctaggtctccttcttataggacaaaggctaagtctcatggggctctgtctgattctaagtctgtgtctaatctgtctgaggctagggctcagattgctgagccttctacatctaggtattttccttcttcttgtctggatctgaattctcctacatctcctacacctacaaggcattctgatacaaggacactgctgtctccttctgggaggaataataggaatgaggggacactggattctaggaggacaacaacaaggcattctaagacaatggaggagctgaagctgcctgagcatatggattcttctcattctcattctctgtctgctcctcatgagtctttttcttatgggctggggtatacatctcctttttcttctcagcagaggcctcataggcattctatgtatgtgacaagggataaggtgagggctaaggggctggatgggtctctgtctattgggcaggggatggctgctagggctaattctctgcagctgctgtctcctcagcctggggagcagctgcctcctgagatgacagtggctaggtcttctgtgaaggagacatctagggaggggacatcttcttttcatacaaggcagaagtctgaggggggggtgtatcatgatcctcattctgatgatgggacagctcctaaggagaataggcatctgtataatgatcctgtgcctaggagggtggggtctttttatagggtgccttctcctaggcctgataattcttttcatgagaataatgtgtctacaagggtgtcttctctgccttctgagtcttcttctgggacaaatcattctaagaggcagcctgcttttgatccttggaagtctcctgagaatatttctcattctgagcagctgaaggagaaggagaagcaggggttttttaggtctatgaagaagaagaagaagaagtctcagacagtgcctaattctgattctcctgatctgctgacactgcagaagtctattcattctgcttctacaccttcttctaggcctaaggagtggaggcctgagaagatttctgatctgcagacacagtctcagcctctgaagtctctgaggaagctgctgcatctgtcttctgcttctaatcatcctgcttcttctgatcctaggtttcagcctctgacagctcagcagacaaagaattctttttctgagattaggattcatcctctgtctcaggcttctggggggtcttctaatattaggcaggagcctgctcctaaggggaggcctgctctgcagctgcctgatggggggtgtgatgggaggaggcagaggcatcattctgggcctcaggataggaggtttatgctgaggacaacagagcagcagggggagtatttttgttgtggggatcctaagaagcctcatacaccttgtgtgcctaatagggctctgcataggcctatttcttctcctgctccttatcctgtgctgcaggtgagggggacatctatgtgtcctacactgcaggtgagggggacagatgctttttcttgtcctacacagcagtctgggttttctttttttgtgaggcatgtgatgagggaggctctgattcatagggctcaggtgaatcaggctgctctgctgacatatcatgagaatgctgctctgacagggaag

SEQ ID NO: 18 - DNA, 3090 bp, synthetic construct
  source 1..3090: mol_type = other DNA
atgaagattccaaatattgggaatgtgatgaataagtttgagattctgggggtggtgggggagggggcatatggggtggtgctgaagtgtaggcataaggagacacatgagattgtggcaattaagaagtttaaggattcagaggagaatgaggaggtgaaggagacaacactgagggagctgaagatgctgaggacactgaagcaggagaatattgtggagctgaaggaggcatttaggaggagggggaagctgtatctggtgtttgagtatgtggagaagaatatgctggagctgctggaggagatgccaaatggggtgccaccagagaaggtgaagtcatatatttatcagctgattaaggcaattcattggtgtcataagaatgatattgtgcatagggatattaagccagagaatctgctgatttcacataatgatgtgctgaagctgtgtgattttgggtttgcaaggaatctgtcagaggggaataatgcaaattatacagagtatgtggcaacaaggtggtataggtcaccagagctgctgctgggggcaccatatgggaagtcagtggatatgtggtcagtggggtgtattctgggggagctgtcagatgggcagccactgtttccaggggagtcagagattgatcagctgtttacaattcagaaggtgctggggccactgccatcagagcagatgaagctgttttattcaaatccaaggtttcatgggctgaggtttccagcagtgaatcatccacagtcactggagaggaggtatctggggattctgaattcagtgctgctggatctgatgaagaatctgctgaagctggatccagcagataggtatctgacagagcagtgtctgaatcatccaacatttcagacacagaggctgctggataggtcaccatcaaggtcagcaaagaggaagccatatcatgtggagtcatcaacactgtcaaataggaatcaggcagggaagtcaacagcactgcagtcacatcataggtcaaattcaaaggatattcagaatctgtcagtggggctgccaagggcagatgaggggctgccagcaaatgagtcatttctgaatgggaatctggcaggggcatcactgtcaccactgcatacaaagacatatcaggcatcatcacagccagggtcaacatcaaaggatctgacaaataataatattccacatctgctgtcaccaaaggaggcaaagtcaaagacagagtttgattttaatattgatccaaagccatcagaggggccagggacaaagtatctgaagtcaaattcaaggtcacagcagaataggcattcatttatggagtcatcacagtcaaaggcagggacactgcagccaaatgagaagcagtcaaggcattcatatattgatacaattccacagtcatcaaggtcaccatcatataggacaaaggcaaagtcacatggggcactgtcagattcaaagtcagtgtcaaatctgtcagaggcaagggcacagattgcagagccatcaacatcaaggtattttccatcatcatgtctggatctgaattcaccaacatcaccaacaccaacaaggcattcagatacaaggacactgctgtcaccatcagggaggaataataggaatgaggggacactggattcaaggaggacaacaacaaggcattcaaagacaatggaggagctgaagctgccagagcatatggattcatcacattcacattcactgtcagcaccacatgagtcattttcatatgggctggggtatacatcaccattttcatcacagcagaggccacataggcattcaatgtatgtgacaagggataaggtgagggcaaaggggctggatgggtcactgtcaattgggcaggggatggcagcaagggcaaattcactgcagctgctgtcaccacagccaggggagcagctgccaccagagatgacagtggcaaggtcatcagtgaaggagacatcaagggaggggacatcatcatttcatacaaggcagaagtcagaggggggggtgtatcatgatccacattcagatgatgggacagcaccaaaggagaataggcatctgtataatgatccagtgccaaggagggtggggtcattttatagggtgccatcaccaaggccagataattcatttcatgagaataatgtgtcaacaagggtgtcatcactgccatcagagtcatcatcagggacaaatcattcaaagaggcagccagcatttgatccatggaagtcaccagagaatatttcacattcagagcagctgaaggagaaggagaagcaggggttttttaggtcaatgaagaagaagaagaagaagtcacagacagtgccaaattcagattcaccagatctgctgacactgcagaagtcaattcattcagcatcaacaccatcatcaaggccaaaggagtggaggccagagaagatttcagatctgcagacacagtcacagccactgaagtcactgaggaagctgctgcatctgtcatcagcatcaaatcatccagcatcatcagatccaaggtttcagccactgacagcacagcagacaaagaattcattttcagagattaggattcatccactgtcacaggcatcaggggggtcatcaaatattaggcaggagccagcaccaaaggggaggccagcactgcagctgccagatggggggtgtgatgggaggaggcagaggcatcattcagggccacaggataggaggtttatgctgaggacaacagagcagcagggggagtatttttgttgtggggatccaaagaagccacatacaccatgtgtgccaaatagggcactgcataggccaatttcatcaccagcaccatatccagtgctgcaggtgagggggacatcaatgtgtccaacactgcaggtgagggggacagatgcattttcatgtccaacacagcagtcagggttttcattttttgtgaggcatgtgatgagggaggcactgattcatagggcacaggtgaatcaggcagcactgctgacatatcatgagaatgcagcactgacagggaag

SEQ ID NO: 19 - DNA, 3090 bp, synthetic construct
  source 1..3090: mol_type = other DNA
atgaagataccaaatataggtaatgtaatgaataagtttgaaatactaggtgtagtaggtgaaggtgcatatggtgtagtactaaagtgtaggcataaggaaacacatgaaatagtagcaataaagaagtttaaggattcagaagaaaatgaagaagtaaaggaaacaacactaagggaactaaagatgctaaggacactaaagcaagaaaatatagtagaactaaaggaagcatttaggaggaggggtaagctatatctagtatttgaatatgtagaaaagaatatgctagaactactagaagaaatgccaaatggtgtaccaccagaaaaggtaaagtcatatatatatcaactaataaaggcaatacattggtgtcataagaatgatatagtacatagggatataaagccagaaaatctactaatatcacataatgatgtactaaagctatgtgattttggttttgcaaggaatctatcagaaggtaataatgcaaattatacagaatatgtagcaacaaggtggtataggtcaccagaactactactaggtgcaccatatggtaagtcagtagatatgtggtcagtaggttgtatactaggtgaactatcagatggtcaaccactatttccaggtgaatcagaaatagatcaactatttacaatacaaaaggtactaggtccactaccatcagaacaaatgaagctattttattcaaatccaaggtttcatggtctaaggtttccagcagtaaatcatccacaatcactagaaaggaggtatctaggtatactaaattcagtactactagatctaatgaagaatctactaaagctagatccagcagataggtatctaacagaacaatgtctaaatcatccaacatttcaaacacaaaggctactagataggtcaccatcaaggtcagcaaagaggaagccatatcatgtagaatcatcaacactatcaaataggaatcaagcaggtaagtcaacagcactacaatcacatcataggtcaaattcaaaggatatacaaaatctatcagtaggtctaccaagggcagatgaaggtctaccagcaaatgaatcatttctaaatggtaatctagcaggtgcatcactatcaccactacatacaaagacatatcaagcatcatcacaaccaggttcaacatcaaaggatctaacaaataataatataccacatctactatcaccaaaggaagcaaagtcaaagacagaatttgattttaatatagatccaaagccatcagaaggtccaggtacaaagtatctaaagtcaaattcaaggtcacaacaaaataggcattcatttatggaatcatcacaatcaaaggcaggtacactacaaccaaatgaaaagcaatcaaggcattcatatatagatacaataccacaatcatcaaggtcaccatcatataggacaaaggcaaagtcacatggtgcactatcagattcaaagtcagtatcaaatctatcagaagcaagggcacaaatagcagaaccatcaacatcaaggtattttccatcatcatgtctagatctaaattcaccaacatcaccaacaccaacaaggcattcagatacaaggacactactatcaccatcaggtaggaataataggaatgaaggtacactagattcaaggaggacaacaacaaggcattcaaagacaatggaagaactaaagctaccagaacatatggattcatcacattcacattcactatcagcaccacatgaatcattttcatatggtctaggttatacatcaccattttcatcacaacaaaggccacataggcattcaatgtatgtaacaagggataaggtaagggcaaagggtctagatggttcactatcaataggtcaaggtatggcagcaagggcaaattcactacaactactatcaccacaaccaggtgaacaactaccaccagaaatgacagtagcaaggtcatcagtaaaggaaacatcaagggaaggtacatcatcatttcatacaaggcaaaagtcagaaggtggtgtatatcatgatccacattcagatgatggtacagcaccaaaggaaaataggcatctatataatgatccagtaccaaggagggtaggttcattttatagggtaccatcaccaaggccagataattcatttcatgaaaataatgtatcaacaagggtatcatcactaccatcagaatcatcatcaggtacaaatcattcaaagaggcaaccagcatttgatccatggaagtcaccagaaaatatatcacattcagaacaactaaaggaaaaggaaaagcaaggtttttttaggtcaatgaagaagaagaagaagaagtcacaaacagtaccaaattcagattcaccagatctactaacactacaaaagtcaatacattcagcatcaacaccatcatcaaggccaaaggaatggaggccagaaaagatatcagatctacaaacacaatcacaaccactaaagtcactaaggaagctactacatctatcatcagcatcaaatcatccagcatcatcagatccaaggtttcaaccactaacagcacaacaaacaaagaattcattttcagaaataaggatacatccactatcacaagcatcaggtggttcatcaaatataaggcaagaaccagcaccaaagggtaggccagcactacaactaccagatggtggttgtgatggtaggaggcaaaggcatcattcaggtccacaagataggaggtttatgctaaggacaacagaacaacaaggtgaatatttttgttgtggtgatccaaagaagccacatacaccatgtgtaccaaatagggcactacataggccaatatcatcaccagcaccatatccagtactacaagtaaggggtacatcaatgtgtccaacactacaagtaaggggtacagatgcattttcatgtccaacacaacaatcaggtttttcattttttgtaaggcatgtaatgagggaagcactaatacatagggcacaagtaaatcaagcagcactactaacatatcatgaaaatgcagcactaacaggtaag

SEQ ID NO: 20 - DNA, 3828 bp, synthetic construct
  source 1..3828: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccgccaccatgaagattcctaacattggtaatgtgatgaataaatttgagatccttggggttgtaggtgaaggagcctatggagttgtacttaaatgcagacacaaggaaacacatgaaattgtggcgatcaagaaattcaaggacagtgaagaaaatgaagaagtcaaagaaacgactttacgagagcttaaaatgcttcggactctcaagcaggaaaacattgtggagttgaaggaagcatttcgtcggaggggaaagttgtacttggtgtttgagtatgttgaaaaaaatatgctcgaattgctggaagaaatgccaaatggagttccacctgagaaagtaaaaagctacatctatcagctaatcaaggctattcactggtgccataagaatgatattgtccatcgagatataaaaccagaaaatctcttaatcagccacaatgatgtcctaaaactgtgtgactttggttttgctcgtaatctgtcagaaggcaataatgctaattacacagagtacgttgccaccagatggtatcggtccccagaactcttacttggcgctccctatggaaagtccgtggacatgtggtcggtgggctgtattcttggggagcttagcgatggacagcctttatttcctggagaaagtgaaattgaccaactttttactattcagaaggtgctaggaccacttccatctgagcagatgaagcttttctacagtaatcctcgcttccatgggctccggtttccagctgttaaccatcctcagtccttggaaagaagataccttggaattttgaatagtgttctacttgacctaatgaagaatttactgaagttggacccagctgacagatacttgacagaacagtgtttgaatcaccctacatttcaaacccagagacttctggatcgttctccttcaaggtcagcaaaaagaaaaccttaccatgtggaaagcagcacattgtctaatagaaaccaagccggcaaaagtactgctttgcagtctcaccacagatctaacagcaaggacatccagaacctgagtgtaggcctgccccgggctgacgaaggtctccctgccaatgaaagcttcctaaatggaaaccttgctggagctagtcttagtccactgcacaccaaaacctaccaagcaagcagccagcctgggtctaccagcaaagatctcaccaacaacaacataccacaccttcttagcccaaaagaagccaagtcaaaaacagagtttgattttaatattgacccaaagccttcagaaggcccagggacaaagtacctcaagtcaaacagcagatctcagcagaaccgccactcattcatggaaagctctcaaagcaaagctgggacactgcagcccaatgaaaagcagagtcggcatagctatattgacacaattccccagtcctctaggagtccctcctacaggaccaaggccaaaagccatggggcactgagtgactccaagtctgtgagcaacctttctgaagccagggcccaaattgcggagcccagtaccagtaggtacttcccatctagctgcttagacttgaattctcccaccagcccaacccccaccagacacagtgacacgagaactttgctcagcccttctggaagaaataaccgaaatgagggaacgctggactcacgtcgaaccacaaccagacattctaagacgatggaggaattgaagctgccggagcacatggacagtagccattcccattcactgtctgcacctcacgaatctttttcttatggactgggctacaccagccccttttcttcccagcaacgtcctcataggcattctatgtatgtgacccgtgacaaagtgagagccaagggcttggatggaagcttgagcatagggcaagggatggcagctagagccaacagcctgcaactcttgtcaccccagcctggagaacagctccctccagagatgactgtggcaagatcttcggtcaaagagacctccagagaaggcacctcttccttccatacacgccagaagtctgagggtggagtgtatcatgacccacactctgatgatggcacagcccccaaagaaaatagacacctatacaatgatcctgtgccaaggagagttggtagcttttacagagtgccatctccacgtccagacaattctttccatgaaaataatgtgtcaactagagtttcttctctaccatcagagagcagttctggaaccaaccactcaaaaagacaaccagcattcgatccatggaaaagtcctgaaaatattagtcattcagagcaactcaaggaaaaagagaagcaaggatttttcaggtcaatgaaaaagaaaaagaagaaatctcaaacagtacccaattccgacagccctgatcttctgacgttgcagaaatccattcattctgctagcactccaagcagcagaccaaaggagtggcgccccgagaagatctcagatctgcagacccaaagccagccattaaaatcactgcgcaagttgttacatctctcttcggcctcaaatcacccggcttcctcagatccccgcttccagcccttaacagctcaacaaaccaaaaattccttctcagaaattcggattcaccccctgagccaggcctctggcgggagcagcaacatccggcaggaacccgcaccgaagggcaggccagccctccagctgccaggtcagatggatcctggttggcatgtgtcctctgtgaccaggagtgccacagagggcccttcctactctgaacagctgggtgccaaaagtgggccaaatgggcacccctataacagaacaaatcgctcacgaatgccaaatctgaatgatttaaaagagacagccttgtaagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

(19) United States
(12) Patent Application Publication
(10) Pub. No.: US 2025/0011810 A1
(43) Pub. Date: Jan. 9, 2025

(54) RECOMBINANT AAV FORMULATIONS

(71) Applicant: Ultragenyx Pharmaceutical Inc., Novato, CA (US)

(72) Inventors: Julie Yu Wei, Lexington, MA (US); Sharyl Lynne Fyffe-Maricich, Novato, CA (US); James D. Maratt, Belmont, MA (US); James C. Warren, Hollis, NH (US)

(21) Appl. No.: 18/684,333

(22) PCT Filed: Aug. 18, 2022
(86) PCT No.: PCT/US2022/075130

§ 371 Date: Feb. 16, 2024

Related U.S. Application Data
(60) Provisional application No. 63/234,408, filed on Aug. 18, 2021.

(51) Int. Cl. C12N 15/86; A61K 9/00; A61K 47/26; A61K 47/34

(52) U.S. Cl. CPC C12N 15/86; A61K 9/0019; A61K 47/26; A61K 47/34; C12N 2750/14132; C12N 2750/14143

(57) ABSTRACT

This present disclosure provides pharmaceutical compositions for delivering recombinant adeno-associated virus (rAAV) particles, generally comprising a buffer, a monovalent salt, a poly hydric alcohol, and a triblock copolymer surfactant.

### CROSS-REFERENCE TO RELATED APPLICATIONS

The present application claims the benefit of and priority to U.S. Provisional Application No. 63/234,408 filed Aug. 18, 2021, the entire content of which is hereby incorporated by reference in its entirety for all purposes.

### SEQUENCE LISTING

The instant application contains a Sequence Listing which has been submitted electronically in XML format and is hereby incorporated by reference in its entirety. Said XML file, created on Aug. 11, 2022, is named ULP_013WO_SL.xml and is 49 kilobytes in size.

### TECHNICAL FIELD OF THE INVENTION

The present disclosure relates generally to compositions comprising recombinant adeno-associated viral vectors, and methods of their use.

### BACKGROUND OF THE INVENTION

Adeno-associated viruses (AAV) are small, non-enveloped virus that packages a linear, single-stranded DNA genome. AAV vectors are able to mediate transfer of a heterologous gene, e.g., a gene encoding a functional protein for gene therapy. Some recombinant AAV (rAAV) vectors are in development for treating various disorders, such as central nervous system disorders. However, delivery of therapeutic agents to the central nervous system is often challenging. For example, in some situations, viral vectors have been observed to transduce only those cells in the vicinity of the injection site or otherwise exhibit very limited transduction efficiency.

The present invention addresses this need by providing compositions for delivery of rAAV, e.g., to the central nervous system.

### SUMMARY OF THE INVENTION

This invention provides compositions comprising recombinant adeno-associated virus (rAAV) and methods of their use. For example, compositions disclosed herein can be used to deliver rAAV vectors for gene therapy, e.g., to the central nervous system.

In one aspect, provided are pharmaceutical compositions comprising:
a) recombinant adeno-associated virus (rAAV) particles;
b) a buffer;
c) a monovalent salt;
d) a polyhydric alcohol; and
e) a triblock copolymer surfactant.

In some embodiments, the pharmaceutical composition has a pH between 7.0 and 7.4, e.g., about 7.2.

In some embodiments, the buffer is a phosphate buffer. In some embodiments, phosphate is present in the composition at a concentration of between 5 mM and 30 mM, e.g., about 10 mM.

In some embodiments, the buffer is a Tris buffer. In some embodiments, Tris is present in the composition at a concentration of between 5 mM and 30 mM, e.g., about 10 mM.

In some embodiments, the polyhydric alcohol is a sugar alcohol, e.g., a sugar alcohol selected from the group consisting of erythritol, glycerol, isomalt, lactitol, maltitol, mannitol, sorbitol, and xylitol.

In some embodiments, the sugar alcohol is sorbitol. In some embodiments, sorbitol is present in the composition at a concentration of at least 1% or at least 5%. In some embodiments, sorbitol is present in the composition at a concentration of between 0.5% and 20%, e.g., between 5% and 10%. In some embodiments, sorbitol is present in the composition at a concentration of about 5%. In some embodiments, sorbitol is present in the composition at a concentration of about 10%.

In some embodiments, the triblock copolymer surfactant is a copolymer of ethylene oxide (EO) and propylene oxide (PO), such as a poloxamer. In some embodiments, the poloxamer is poloxamer 188 (Pluronic® F68). In some embodiments, the poloxamer is present in the composition at a concentration of between about 0.0001% and about 0.001%. In some embodiments, the poloxamer is present in the composition at a concentration of at least 0.0001%, at least 0.0005%, or at least 0.001%. In some embodiments, the poloxamer is present in the composition at a concentration of about 0.0001%. In some embodiments, the poloxamer is present in the composition at a concentration of about 0.001%.

In some embodiments, the polyhydric alcohol is sorbitol and the triblock copolymer surfactant is a poloxamer.

In some embodiments, the monovalent salt is NaCl, KCl, or a combination thereof.

In some embodiments, NaCl is present in the composition at a concentration of between 100 mM and 250 mM, e.g., about 130 mM or about 135 mM.

In some embodiments, the pharmaceutical composition comprises NaCl and KCl.

In some embodiments, KCl is present in the composition at a concentration of between 0.5 mM and 5 mM, e.g., about 2 mM.

In some embodiments, the pharmaceutical composition further comprises one or more divalent salts, e.g., MgCl₂, CaCl₂, or a combination thereof.

In some embodiments, MgCl₂ is present in the composition at a concentration of between 0.5 mM and 5 mM, e.g., about 1 mM.

In some embodiments, CaCl₂ is present in the composition at a concentration of between 0.5 mM and 5 mM, e.g., about 1 mM CaCl₂.

In some embodiments, rAAV particles are present in the composition at a concentration of between 1×10¹⁰ and 2×10¹⁴ GC/mL.

In some embodiments, the rAAV particles comprise an AAV capsid from AAV9, AAV1, AAV2, AAV3, AAV4, AAV5, AAV6, AAV7, AAV8, AAV10, AAV11, AAV12, AAV13, AAVrh10, AAVhu37, or a variant thereof. In some embodiments, the rAAV particles comprise an AAV capsid from AAV9.

For example, in some embodiments, pharmaceutical compositions of the present disclosure comprise:
a) recombinant adeno-associated virus (rAAV) particles;
b) phosphate at a concentration of between 5 mM and 30 mM
c) NaCl at a concentration of between 100 mM and 250 mM;
d) sorbitol at a concentration of between 1% and 10%; and
e) poloxamer at a concentration of between 0.0001% and 0.001%,
wherein the composition has a pH between 7.0 and 7.4.

In some embodiments, pharmaceutical composition of the present disclosure comprise:
a) recombinant adeno-associated virus (rAAV) particles;
b) phosphate at a concentration of about 10 mM;
c) NaCl at a concentration of about 135 mM;
d) sorbitol at a concentration of about 5%; and
e) poloxamer at a concentration of about 0.001%,
wherein the composition has a pH of about 7.2.

In some embodiments, pharmaceutical compositions of the present disclosure comprise:
a) recombinant adeno-associated virus (rAAV) particles;
b) phosphate at a concentration of about 10 mM;
c) NaCl at a concentration of about 135 mM;
d) sorbitol at a concentration of about 10%; and
e) poloxamer at a concentration of about 0.001%,
wherein the composition has a pH of about 7.2.

In some embodiments, pharmaceutical compositions of the present disclosure comprise:
a) recombinant adeno-associated virus (rAAV) particles;
b) Tris at a concentration of between 5 mM and 30 mM;
c) NaCl at a concentration of between 100 mM and 250 mM;
d) KCl at a concentration of between 0.5 mM and 5 mM;
e) MgCl₂ at a concentration of between 0.5 mM and 5 mM;
f) CaCl₂ at a concentration of between 0.5 mM and 5 mM;
d) sorbitol at a concentration of between 1% and 10%; and
e) poloxamer at a concentration of between 0.0001% and 0.001%,
wherein the composition has a pH between 7.0 and 7.4.

In some embodiments, pharmaceutical compositions of the present disclosure comprise:
a) recombinant adeno-associated virus (rAAV) particles;
b) about 10 mM Tris;
c) about 130 mM NaCl;
d) about 2 mM KCl;
e) about 1 mM MgCl₂;
f) about 1 mM CaCl₂;
d) about 5% sorbitol; and
e) about 0.001% poloxamer,
wherein the composition has a pH of about 7.2.

In some embodiments, the poloxamer in a pharmaceutical composition of the present disclosure is poloxamer 188 (Pluronic® F68).

In some embodiments, the rAAV particles comprise an AAV capsid from AAV9, AAV1, AAV2, AAV3, AAV4, AAV5, AAV6, AAV7, AAV8, AAV10, AAV11, AAV12, AAV13, AAVrh10, AAVhu37, or a variant thereof. In some embodiments, the rAAV particles comprise an AAV capsid from AAV9.

In some embodiments, the rAAV particles comprise an AAV capsid and a vector genome packaged therein, wherein said vector genome comprises a partial or complete coding sequence for CDKL5, or a functional fragment or variant thereof.

In some embodiments, the coding sequence for CDKL5 comprises a nucleotide sequence selected from SEQ ID NOs: 12-18 and 19, or a nucleotide sequence at least 95% identical to any of SEQ ID NOs: 12-19.

In some embodiments, the vector genome comprises a nucleotide sequence of SEQ ID NO: 20 or a sequence at least 95% identical thereto.

In some embodiments, the pharmaceutical composition is suitable for intrathecal administration. In some embodiments, the intrathecal administration comprises intraventricular, lumbar, or intra-cisterna magna administration.

In one aspect, provided are methods of delivering rAAV to the central nervous system of a subject, comprising a step of administering to the subject a pharmaceutical composition of as disclosed herein. In some embodiments, the subject is a mammal.

In some embodiments, the step of administering comprises administration by intrathecal administration, e.g., intrathecal administration that comprises intraventricular, lumbar, or intra-cisterna magna administration. In some embodiments, the intrathecal administration comprises intra-cisterna magna administration.

### BRIEF DESCRIPTION OF THE DRAWINGS

The invention can be more completely understood with reference to the following drawings.

FIG. 1 is a bar graph that shows the calculated number of genome copies (GC) per μg of DNA in various brain regions (frontal cortex (“Ctx”), caudal cortex, cerebellum, and brainstem) from brain tissues administered rAAV formulations disclosed herein, as described further in Example 2.

FIGS. 2A-2D depict representative images of tissue sections with staining corresponding to RNA expression of vector genomes (as detected by BaseScope®) in mice administered rAAV formulations disclosed herein, as described further in Example 2. FIGS. 2A, 2B, 2C, and 2D depict images from tissue sections from mice in groups 3, 4, 5, and 6, respectively. (See Example 2.)

FIGS. 2E-2H are pie graphs depicting corresponding results from quantitation of BaseScope signals, indicative of transduction efficiency, in the cortex of mice administered rAAV formulations disclosed herein, as described further in Example 2.

FIG. 3 is a bar graph that depicts the percentage area (in cortex tissue) where most of the cells have at least 1 genome copy, in mice administered rAAV formulations disclosed herein, as described further in Example 2.

FIGS. 4A-4D depict representative images of tissue sections with staining corresponding to RNA expression of CDKL5 (as detected by RNAScope®) in mice administered in rAAV (in this case, AAV9-SYN-CDKL5) formulations disclosed herein, as described further in Example 2. FIGS. 4A, 4B, 4C, and 4D depict results from mice in groups 3, 4, 5, and 6, respectively. (See Example 2.)

FIG. 5 depicts a Western Blot stained for CDKL5 protein expression in caudal cortex tissue from mice administered rAAV formulations (in this case, AAV9-SYN-CDKL5) disclosed herein, as described further in Example 2. Staining for GADPH protein expression is shown as a loading control.

### DETAILED DESCRIPTION OF CERTAIN EMBODIMENTS OF THE INVENTION

Unless otherwise noted, technical terms are used according to conventional usage. Definitions of common terms in molecular biology may be found in Benjamin Lewin, Genes V, published by Oxford University Press, 1994 (ISBN 0-19-854287-9): Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

About: Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 13 recognized serotypes of AAV (AAV1-13).

Administration/Administer: To provide or give a subject an agent, such as a therapeutic agent (e.g., a recombinant AAV), by any effective route. Exemplary routes of administration include, but are not limited to, injection (such as subcutaneous, intramuscular, intradermal, intraperitoneal, intrathecal, intracerebroventricular, or intravenous administration), oral, intraductal, sublingual, rectal, transdermal, intranasal, vaginal and inhalation routes.

Between: Unless otherwise noted, where the term “between” is used to refer to a numerical range, the range includes the specified endpoints. For example, the range “between 1 mM and 10 mM” includes 1 mM, 10 mM, and values greater than 1 mM but less than 10 mM.

Block copolymer: “Block copolymers” refer to polymers made up of blocks of different polymerized monomers. “Diblock copolymers” have two distinct blocks, whereas “triblock copolymers” have three distinct blocks.

Coding Sequence: A “coding sequence” means the nucleotide sequence encoding a polypeptide in vitro or in vivo when operably linked to appropriate regulatory sequences. The coding sequence may or may not include regions preceding and following the coding region, e.g., 5′ untranslated (5′ UTR) and 3′ untranslated (3′ UTR) sequences, as well as intervening sequences (introns) between individual coding segments (exons).

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

Enhancer: A nucleic acid sequence that increases the rate of transcription by increasing the activity of a promoter.

Intrathecal: As used herein, “intrathecal” administration refers to administration into the intrathecal space that holds cerebrospinal fluid. Examples of intrathecal administration methods include administration via injection into the spinal cord (e.g. lumbar-intrathecal), into the ventricles (e.g., intracerebroventricular (ICV)), or into subarachnoid spaces (e.g., into the cisterna magna (intra-cisterna magna (ICM)).

Intron: A stretch of DNA within a gene that does not contain coding information for a protein. Introns are removed before translation of a messenger RNA.

Inverted terminal repeat (ITR): Symmetrical nucleic acid sequences in the genome of adeno-associated viruses required for efficient replication. ITR sequences are located at each end of the AAV DNA genome. The ITRs serve as the origins of replication for viral DNA synthesis and are state-of-the-art cis components for generating AAV integrating vectors.

Isolated: An “isolated” biological component (such as a nucleic acid molecule, protein, virus or cell) has been substantially separated or purified away from other biological components in the cell or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Polyhydric alcohol: Polyhydric alcohols refer to organic compounds that have more than one hydroxyl (—OH) group, including dihydric alcohols (which include two hydroxyl groups) and alcohols having more than two hydroxyl groups.

Promoter: A region of DNA that directs/initiates transcription of a nucleic acid (e.g., a gene). A promoter includes necessary nucleic acid sequences near the start site of transcription. Many promoter sequences are known to the person skilled in the art and even a combination of different promoter sequences in artificial nucleic acid molecules is possible. As used herein, a gene-specific endogenous promoter refers to a native promoter element that regulates expression of the endogenous gene of interest.

Purified: The term “purified” does not require absolute purity: rather, it is intended as a relative term. Thus, for example, a purified peptide, protein, virus, or other active compound is one that is isolated in whole or in part from naturally associated proteins and other contaminants. In certain embodiments, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

“Recombinant AAV” (rAAV) is described, e.g., in Wang et al., “Adeno-associated virus vector as a platform for gene therapy delivery.” Nat Rev Drug Discov. 2019 May: 18 (5): 358-378 and Samulski et al., “AAV-Mediated Gene Therapy for Research and Therapeutic Purposes.” Annu Rev Virol. 2014 November: 1 (1): 427-51. In the present disclosure, rAAV is used interchangeably with “rAAV particle.”

Sequence identity: The identity or similarity between two or more nucleic acid sequences, or two or more amino acid sequences, is expressed in terms of the identity or similarity between the sequences. Sequence identity can be measured in terms of percentage identity: the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981: Needleman & Wunsch, J. Mol. Biol. 48:443, 1970: Pearson & Lipman, Proc. Natl. Acad. Sci. USA 85:2444, 1988: Higgins & Sharp, Gene. 73:237-44, 1988: Higgins & Sharp, CABIOS 5:151-3, 1989; Corpet et al., Nuc. Acids Res. 16:10881-90, 1988: Huang et al. Computer Appls. in the Biosciences 8, 155-65, 1992; and Pearson et al., Meth. Mol. Rio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

Serotype: A group of closely related microorganisms (such as viruses) distinguished by a characteristic set of antigens.

Stuffer sequence: Refers to a sequence of nucleotides contained within a larger nucleic acid molecule (such as a vector) that is typically used to create desired spacing between two nucleic acid features (such as between a promoter and a coding sequence), or to extend a nucleic acid molecule so that it is of a desired length. Stuffer sequences do not contain protein coding information and can be of unknown/synthetic origin and/or unrelated to other nucleic acid sequences within a larger nucleic acid molecule.

Subject: Living multi-cellular vertebrate organisms, a category that includes human and non-human mammals. In some embodiments, the subject is a mammal. In some embodiments, the subject is a human. In one embodiment, the human subject is an adult subject, i.e., a human subject greater than 18 years old. In one embodiment, the human subject is a pediatric subject, i.e., a human subject of ages 0-18 years old inclusive. In some embodiments, the subject (e.g., human subject) has been administered a corticosteroid. In some embodiments, the subject (e.g., human subject) has been administered an IgG-degrading protease. In some embodiments, the subject (e.g., human subject) has been administered a corticosteroid and has also been administered an IgG-degrading protease.

Synthetic: Produced by artificial means in a laboratory, for example a synthetic nucleic acid can be chemically synthesized in a laboratory.

Untranslated region (UTR): A typical mRNA contains a 5′ untranslated region (5′ UTR) and a 3′ untranslated region (3′ UTR) upstream and downstream, respectively, of the coding region (see Mignone F. et. al., (2002) Genome Biol 3: REVIEWS0004).

Therapeutically effective amount: A quantity of a specified pharmaceutical or therapeutic agent (e.g., a recombinant AAV) sufficient to achieve a desired effect in a subject, or in a cell, being treated with the agent. The effective amount of the agent will be dependent on several factors, including, but not limited to the subject or cells being treated, and the manner of administration of the therapeutic composition.

Treating, ameliorating, or preventing a disease: “Treating” refers to a therapeutic intervention that ameliorates a sign or symptom of a disease or pathological condition after it has begun to develop. “Ameliorating” refers to the reduction in the number or severity of signs or symptoms of a disease. “Preventing” a disease refers to inhibiting the full development of a disease.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector. The term “vector” may also be used in the general sense of an agent that carries genetic information or material, for example, to introduce the genetic material to a cell or organism. Persons skilled in the art will readily appreciate the generic uses of the term “vector” as an agent that carries genetic information or material as well as the more specific uses of the term “vector” referring to a plasmid or a vector genome within a rAAV particle.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### I. Pharmaceutical Compositions

In many embodiments, provided compositions are in liquid form, e.g., as a solution comprising various components as described herein.

### Buffers and pH

Provided compositions generally comprise a buffer/carrier suitable for infusion in human subjects. The buffer/carrier should include a component that prevents the rAAV from sticking to the infusion tubing but does not interfere with the rAAV binding activity in vivo. A variety of pH buffering agents may be suitable for use with the presently disclosed compositions.

Non-limiting examples of suitable buffering agents include, e.g., phosphate and Tris (tris(hydroxymethyl) aminomethane). For example, in some embodiments, the composition comprises phosphate, e.g., phosphate is present in the composition at a concentration between 5 mM and 30 mM phosphate. In some embodiments, phosphate is present in the composition at a concentration of between 5 mM and 29 mM, between 5 mM and 28 mM, between 5 mM and 27 mM, between 5 mM and 26 mM, between 5 mM and 25 mM, between 5 mM and 24 mM, between 5 mM and 23 mM, between 5 mM and 22 mM, between 5 mM and 21 mM, between 5 mM and 20 mM, between 5 mM and 19 mM, between 5 mM and 18 mM, between 5 mM and 17 mM, between 5 mM and 16 mM, between 5 mM and 15 mM, between 5 mM and 14 mM phosphate, between 5 mM and 13 mM, between 5 mM and 12 mM, between 5 mM and 11 mM, 5 between mM and 10 mM, between 5 mM and 9 mM, between 5 mM and 8 mM, between 5 mM and 7 mM, or between 5 mM and 6 mM. In some embodiments, phosphate is present in the composition at a concentration of between 6 mM and 30 mM, between 7 mM and 30 mM, between 8 mM and 30 mM, between 9 mM and 30 mM, between 10 mM and 30 mM, between 11 mM and 30 mM, between 12 mM and 30 mM, between 13 mM and 30 mM, between 14 mM and 30 mM, between 15 mM and 30 mM, between 16 mM and 30 mM, between 17 mM and 30 mM, between 18 mM and 30 mM, between 19 mM and 30 mM, between 20 mM and 30 mM, between 21 mM and 30 mM, between 22 mM and 30 mM, between 23 mM and 30 mM, between 24 mM and 30 mM, between 25 mM and 30 mM, between 26 mM and 30 mM, between 27 mM and 30 mM, between 28 mM and 30 mM, or between 29 mM and 30 mM. In some embodiments, phosphate is present in the composition at a concentration of at least 5 mM, e.g., about 10 mM, about 15 mM, or about 20 mM. In some embodiments, the phosphate is present in the composition at a concentration of about 10 mM.

In some embodiments, Tris is present in the composition, e.g., at a concentration of between 5 mM and 30 mM. In some embodiments, Tris is present in the composition at a concentration of between 5 mM and 29 mM, between 5 mM and 28 mM, between 5 mM and 27 mM, between 5 mM and 26 mM, between 5 mM and 25 mM, between 5 mM and 24 mM, between 5 mM and 23 mM, between 5 mM and 22 mM, between 5 mM and 21 mM, between 5 mM and 20 mM, between 5 mM and 19 mM, between 5 mM and 18 mM, between 5 mM and 17 mM, between 5 mM and 16 mM, between 5 mM and 15 mM, between 5 mM and 14 mM, between 5 mM and 13 mM, between 5 mM and 12 mM, between 5 mM and 11 mM, between 5 mM and 10 mM, 5 mM-9 mM Tris, between 5 mM and 8 mM, between 5 mM and 7 mM, or between 5 mM and 6 mM. In some embodiments, Tris is present in the composition at a concentration of between 6 mM and 30 mM, between 7 mM and 30 mM, between 8 mM and 30 mM, between 9 mM and 30 mM, between 10 mM and 30 mM, between 11 mM and 30 mM, between 12 mM and 30 mM, between 13 mM and 30 mM, between 14 mM and 30 mM, between 15 mM and 30 mM, between 16 mM and 30 mM, between 17 mM and 30 mM, between 18 mM and 30 mM, between 19 mM and 30 mM, between 20 mM and 30 mM, between 21 mM and 30 mM, between 22 mM and 30 mM, between 23 mM and 30 mM, between 24 mM and 30 mM, between 25 mM and 30 mM, between 26 mM and 30 mM, between 27 mM and 30 mM, between 28 mM and 30 mM, or between 29 mM and 30 mM Tris. In some embodiments, Tris is present in the composition at a concentration of at least 5 mM, e.g., about 10 mM, about 15 mM, or about 20 mM. In some embodiments, Tris is present in the composition at a concentration of about 10 mM.

The pH of provided compositions are generally between 6.5 and 8.5, e.g., between 7.0 and 8.5, between 7.5 and 8, or between 7.0 and 7.4. In some embodiments, the composition has a pH of about 7.2.

### Polyhydric Alcohols

Non-limiting examples of suitable polyhydric alcohols include, e.g., polyethylene glycol, propylene glycol, and sugar alcohols.

In certain embodiments, the polyhydric alcohol is a sugar alcohol. In some embodiments, the polyhydric alcohol is a sugar alcohol selected from the group consisting of erythritol, glycerol, isomalt, lactitol, maltitol, mannitol, sorbitol, and xylitol. In some embodiments, the sugar alcohol is sorbitol. In some embodiments, the polyhydric alcohol is a sugar alcohol in its hydrogenated forms of mono-(erythritol, xylitol, sorbitol, mannitol), di-(lactitol, isomalt, maltitol), or polysaccharides forms.

In some embodiments, the polyhydric alcohol is present in the formulation at a range of 0.5% to 20%. (Unless otherwise noted, percentages of polyhydric alcohols in liquid compositions throughout this disclosure refer to weight/volume percentages. 1 g/100 mL is equivalent to 1% (w/v).) In some embodiments, the polyhydric alcohol is present in the formulation at a range of 0.5% to 19%, 0.5% to 18%, 0.5% to 17%, 0.5% to 16%, 0.5% to 15%, 0.5% to 14%, 0.5% to 13%, 0.5% to 12%, 0.5% to 11%, 0.5% to 10%, 0.5% to 9%, 0.5% to 8%, 0.5% to 7%, 0.5% to 6%, 0.5% to 5%, 0.5% to 4%, 0.5% to 3%, 0.5% to 2%, 0.5% to 1%, 0.5% to 0.9%, 0.5% to 0.8%, 0.5% to 0.7%, or 0.5% to 0.6%. In some embodiments, the polyhydric alcohol is present in the formulation at a range of 0.6% to 20%, 0.7% to 20%, 0.8% to 20%, 0.9% to 20%, 1% to 20%, 2% to 20%, 3% to 20%, 4% to 20%, 5% to 20%, 6% to 20%, 7% to 20%, 8% to 20%, 9% to 20%, 10% to 20%, 11% to 20%, 12% to 20%, 13% to 20%, 14% to 20%, 15% to 20%, 16% to 20%, 17% to 20%, 18% to 20%, or 19% to 20%. In some embodiments, polyhydric alcohol is present in the formulation at a range of 1% to 10%. In some embodiments, polyhydric alcohol is present in the formulation at about 1%, about 2%, about 3%, about 4%, about 5%, about 6%, about 7%, about 8%, about 9%, or about 10%. In some embodiments, polyhydric alcohol is present in the formulation at about 5% or about 10%.

In some exemplary embodiments, the composition comprises sorbitol. In some embodiments, sorbitol is present in the formulation at a range of 0.5% to 20%. In some embodiments, sorbitol is present in the formulation at a range of 0.5% to 19%, 0.5% to 18%, 0.5% to 17%, 0.5% to 16%, 0.5% to 15%, 0.5% to 14%, 0.5% to 13%, 0.5% to 12%, 0.5% to 11%, 0.5% to 10%, 0.5% to 9%, 0.5% to 8%, 0.5% to 7%, 0.5% to 6%, 0.5% to 5%, 0.5% to 4%, 0.5% to 3%, 0.5% to 2%, 0.5% to 1%, 0.5% to 0.9%, 0.5% to 0.8%, 0.5% to 0.7%, or 0.5% to 0.6%. In some embodiments, sorbitol is present in the formulation at a range of 0.6% to 20%, 0.7% to 20%, 0.8% to 20%, 0.9% to 20%, 1% to 20%, 2% to 20%, 3% to 20%, 4% to 20%, 5% to 20%, 6% to 20%, 7% to 20%, 8% to 20%, 9% to 20%, 10% to 20%, 11% to 20%, 12% to 20%, 13% to 20%, 14% to 20%, 15% to 20%, 16% to 20%, 17% to 20%, 18% to 20%, or 19% to 20%. In some embodiments, sorbitol is present in the formulation at a range of 1% to 10%. In some embodiments, sorbitol is present in the formulation at about 1%, about 2%, about 3%, about 4%, about 5%, about 6%, about 7%, about 8%, about 9%, or about 10%. In some embodiments, sorbitol is present in the formulation at about 5% or about 10%.

### Tri-Block Copolymer Surfactants

Non-limiting examples of suitable tri-block copolymer surfactants include non-ionic triblock copolymer surfactants, such as copolymers of poly(ethylene oxide) (PEO) and poly(propylene oxide) (PPO), such as the poloxamers. Poloxamers are triblock copolymers of composed of a central hydrophobic chain of poly(propylene oxide) (PPO) flanked by two hydrophilic chains of poly(ethylene oxide) (PEO). Poloxamers typically exhibit a molecular weight range of from about 1,000 to about 15,000.

In some embodiments, the tri-block copolymer surfactant is a poloxamer, e.g., poloxamer 188 (Pluronic F68®).

In some embodiments, the triblock copolymer surfactant (e.g., poloxamer) is present in the composition at a concentration of at least 0.0001%, at least 0.0005%, or at least 0.001%. (Unless otherwise noted, percentages of triblock copolymer surfactants (e.g., poloxamer) in liquid compositions throughout this disclosure refer to weight/volume percentages. 1 g/100 mL is equivalent to 1% (w/v).) In some embodiments, the triblock copolymer surfactant (e.g., poloxamer) is present in the composition at a concentration of between 0.0001% and about 0.001%. In some embodiments, the triblock copolymer surfactant (e.g., poloxamer) is present in the composition at a concentration of between 0.0002% and 0.001%, between 0.0003% and 0.001%, between 0.0004% and 0.001%, between 0.0005% and 0.001%, between 0.0006% and 0.001%, between 0.0007% and 0.001%, between 0.0008% and 0.001%, between 0.0009% and 0.001%, between 0.0001% and 0.0009%, between 0.0001% and 0.0008%, between 0.0001% and 0.0007%, between 0.0001% and 0.0006%, between 0.0001% and 0.0005%, between 0.0001% and 0.0004%, between 0.0001% and 0.0003%, or between 0.0001% and 0.0002%.

### Salts

Provided compositions may comprise physiologically compatible salts or mixture of salts. In some embodiments, any included salts are adjusted to an ionic strength equivalent to about 100 mM sodium chloride (NaCl) to about 250 mM sodium chloride, or a physiologically compatible salt adjusted to an equivalent ionic concentration.

For example, provided compositions may comprise one or more salts, e.g., one or more monovalent salts and/or one or more divalent salts. For example, provided compositions may comprise a monovalent salt selected from the group consisting of NaCl, KCl, or a combination thereof. For example, in some embodiments, NaCl is present in the composition at a concentration between 100 mM and 250 mM, between 100 mM and 240 mM, between 100 mM and 230 mM, between 100 mM and 220 mM, between 100 mM and 210 mM, between 100 mM and 200 mM NaCl, between 100 mM and 190 mM, between 100 mM and 180 mM, between 100 mM and 170, between 100 mM and 160 mM, between 100 mM and 150 mM, between 100 mM and 140 mM, between 100 mM and 130 mM, between 100 mM and 120 mM, between 100 mM and 110 mM, between 90 mM and 200 mM, between 110 mM and 200 mM, between 120 mM and 200 mM, between 130 mM and 200 mM, between 140 mM and 200 mM, between 150 mM and 200 mM, between 160 mM and 200 mM, between 170 mM and 200 mM, between 180 mM and 200 mM, or between 190 mM and 200 mM.

In some embodiments, NaCl is present in the composition at a concentration of about 130 mM or about 135 mM.

In some embodiments, KCl is present in the composition at a concentration between 0.5 mM and 5 mM, between 0.6 mM and 5 mM, between 0.7 mM and 5 mM, between 0.8 mM and 5 mM, between 0.9 mM and 5 mM, between 1 mM and 5 mM, between 1.5 mM and 5 mM, between 2 mM and 5 mM, between 2.5 mM and 5 mM, between 3 mM and 5 mM, between 3.5 mM and 5 mM, between 4 mM and 5 mM, between 4.5 mM and 5 mM, between 0.5 mM and 4.5 mM, between 0.5 mM and 4 mM, between 0.5 mM and 3.5 mM, between 0.5 mM and 3 mM, between 0.5 mM and 2.5 mM, between 0.5 mM and 2 mM, or between 0.5 mM and 1 mM.

In some embodiments, KCl is present in the composition at a concentration of about 2 mM.

Non-limiting examples of suitable divalent salts include MgCl₂, CaCl₂, or a combination thereof. For example, in some embodiments, MgCl₂ is present in the composition at a concentration between 0.5 mM and 5 mM. between 0.6 mM and 5 mM, between 0.7 mM and 5 mM, between 0.8 mM and 5 mM, between 0.9 mM and 5 mM, between 1 mM and 5 mM, between 1.5 mM and 5 mM, between 2 mM and 5 mM, between 2.5 mM and 5 mM, between 3 mM and 5 mM, between 3.5 mM and 5 mM, between 4 mM and 5 mM, between 4.5 mM and 5 mM, between 0.5 mM and 4.5 mM, between 0.5 mM and 4 mM, between 0.5 mM and 3.5 mM, between 0.5 mM and 3 mM, between 0.5 mM and 2.5 mM, between 0.5 mM and 2 mM, or between 0.5 mM and 1 mM.

In some embodiments, MgCl₂ is present in the composition at a concentration of about 1 mM.

In some embodiments, CaCl₂ is present in the composition at a concentration between 0.5 mM and 5 mM, between 0.6 mM and 5 mM, between 0.7 mM and 5 mM, between 0.8 mM and 5 mM, between 0.9 mM and 5 mM, between 1 mM and 5 mM, between 1.5 mM and 5 mM, between 2 mM and 5 mM, between 2.5 mM and 5 mM, between 3 mM and 5 mM, between 3.5 mM and 5 mM, between 4 mM and 5 mM, or between 4.5 mM and 5 mM, between 0.5 mM and 4.5 mM, between 0.5 mM and 4 mM, between 0.5 mM and 3.5 mM, between 0.5 mM and 3 mM, between 0.5 mM and 2.5 mM, between 0.5 mM and 2 mM, or between 0.5 mM and 1 mM.

In some embodiments, CaCl₂ is present in the composition at a concentration of about 1 mM.

Recombinant AAV (rAAV):

In present disclosure compositions and methods of their use, e.g., to deliver rAAV to the central nervous system of a subject, e.g., for gene therapy, are described.

AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency.

The AAV genome is characterized by two inverted terminal repeats (ITRs) that flank two open reading frames (ORF). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication: the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Days and Berns, Clin Microbiol Rev 21 (4): 583-593, 2008).

The left ORF of AAV contains the Rep gene, which encodes four proteins—Rep78, Rep68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21 (4): 583-593, 2008).

AAV is currently one of the most frequently used viruses for gene therapy. Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. Because of the advantageous features of AAV, the present disclosure contemplates the use of AAV for the recombinant nucleic acid molecules and methods disclosed herein.

AAV possesses several desirable features for a gene therapy vector, including the ability to bind and enter target cells, enter the nucleus, the ability to be expressed in the nucleus for a prolonged period of time, and low toxicity. However, the small size of the AAV genome limits the size of heterologous DNA that can be incorporated. To minimize this problem, AAV vectors have been constructed that do not encode Rep and the integration efficiency element (IEE). The ITRs are retained as they are cis signals required for packaging (Daya and Berns, Clin Microbiol Rev. 21 (4): 583-593, 2008). Methods for producing rAAV suitable for gene therapy are well known in the art (see, for example, U.S. Patent Application NOs. 2012/0100606; 2012/0135515; 2011/0229971; and 2013/0072548; and Ghosh et al., Gene Ther 13 (4): 321-329, 2006), and can be utilized with the recombinant nucleic acid molecules and methods disclosed herein.

In some aspects, the present disclosure provides the use of an rAAV disclosed herein for the treatment of disorder, e.g., disorder affecting the central nervous system, wherein the rAAV includes an AAV capsid and a vector genome packaged therein. In some embodiments, the rAAV contains a packaged genome comprising as operably linked components in 5′ to 3′ order: a 5′-ITR, a promoter sequence, a partial or complete coding sequence for a heterologous gene, or a functional fragment or functional variant thereof, and a 3′-ITR.

In some embodiments, recombinant adeno-associated virus (rAAV) particles are present in the compositions at a concentration between 1×10¹⁰ and 4×10¹⁴ GC/mL, between 1×10¹⁰ and 3×10¹⁴ GC/mL, between 1×10¹⁰ and 2×10¹⁴ GC/mL, 1×10¹⁰ and 1×10¹⁴ GC/mL, between 1×10¹¹ and 1×10¹³ GC/mL, between 1×10¹² and 6×10¹² GC/mL, or between 1×10¹² and 4×10¹² GC/mL.

In some embodiments, the rAAV comprises an AAV capsid and a vector genome packaged therein, wherein said vector genome comprises: (a) a promoter sequence; and (b) a partial or complete coding sequence for a heterologous gene (e.g., a heterologous gene encoding a therapeutic gene product), or a functional fragment or functional variant thereof.

In some embodiments, the packaged vector genome may further comprise a 5′-ITR sequence, an enhancer, an intron, a consensus Kozak sequence, a polyadenylation signal, and/or a 3′-ITR sequence as described herein. In some embodiments, the recombinant vector can further include one or more stuffer nucleic acid sequences. In one embodiment, a stuffer nucleic acid sequence is situated between the intron and the partial or complete coding sequence for the heterologous gene.

In various embodiments described herein, the rAAV comprises an AAV capsid. The AAV capsid can be from an AAV of serotype 1, 2, 3, 4, 5, 6, 7, 8, 9, 10, 11, 12, 13, rh10, or hu37 (i.e., AAV1, AAV2, AAV3, AAV4, AAV5, AAV6, AAV7, AAV8, AAV9, AAV10, AAV11, AAV12, AAVrh10, AAVhu37), as well as any one of the more than 100 variants isolated from human and nonhuman primate tissues. See. e.g., Choi et al., 2005, Curr Gene Ther. 5:299-310, 2005 and Gao et al., 2005, Curr Gene Ther. 5:285-297.

Beyond the aforementioned capsids, also included within the scope of the invention are variant AAV capsids which have been engineered to harbor one or more beneficial therapeutic properties (e.g., improved targeting for select tissues, increased ability to evade the immune response, reduced stimulation of neutralizing antibodies, etc.). Non-limiting examples of such engineered variant capsids are described in U.S. Pat. Nos. 9,506,083, 9,585,971, 9,587,282, 9,611,302, 9,725,485, 9,856,539, 9,909,142, 9,920,097, 10,011,640, 10,081,659, 10,179,176, 10,202,657, 10,214,566, 10,214,785, 10,266,845, 10,294,281, 10,301,648, 10,385,320, and 10,392,632 and in PCT Publication NOs. WO/2017/165859, WO/2018/022905, WO/2018/156654, WO/2018/222503, and WO/2018/226602, the disclosures of which are herein incorporated by reference.

In certain exemplary embodiments, the rAAV administered according to the invention comprises an AAV9 capsid. The AAV9 capsid is a self-assembled AAV capsid composed of multiple AAV9 vp proteins. The AAV9 vp proteins are typically expressed as alternative splice variants encoded by a nucleic acid sequence of SEQ ID NO: 11 or a sequence that is at least 70%, at least 75%, at least 80%, at least 85%, at least 90%, at least 95%, at least 97%, at least 99% identical thereto, which encodes the vp1 amino acid sequence of SEQ ID NO:1 (GenBank Accession: AAS99264). These splice variants result in proteins of different length of SEQ ID NO: 1. As used herein, an AAV9 variant includes, e.g., those described in WO/2016/049230, U.S. Pat. No. 8,927,514, US Patent Publication No. 2015/0344911, and U.S. Pat. No. 8,734,809.

As indicated herein, the rAAV administered according to the invention may comprise, in some embodiments, an AAV9 capsid. However, in other embodiments, another AAV capsid is selected. Tissue specificity may be determined by the capsid type. AAV serotypes which transduce a suitable target (e.g., liver, muscle, lung, or CNS) may be selected as sources for capsids of AAV viral vectors including, e.g., AAV1, AAV2, AAV3, AAV4, AAV5, AAV6. AAV6.2. AAV7, AAV8, AAV9, AAVrh10. AAVrh64R1. AAVrh64R2, AAVrh8. See. e.g., U.S. Patent Publication No. 2007/0036760; US Patent Publication No. 2009/0197338; and EP1310571. See also WO 2003/042397 (AAV7 and other simian AAV), U.S. Pat. Nos. 7,282,199 and 7,790,449 (AAV8). In addition, AAV yet to be discovered, or a recombinant AAV based thereon, may be used as a source for the AAV capsid. These documents also describe other AAV which may be selected for generating AAV and are incorporated by reference. In some embodiments, an AAV capsid for use in the viral vector can be generated by mutagenesis (i.e., by insertions, deletions, or substitutions) of one of the aforementioned AAV capsids or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned capsids.

In some embodiments, the rAAV comprises an AAV capsid and a vector genome packaged therein, wherein said vector genome comprises a partial or complete coding sequence for CDKL5, or a functional fragment or a variant thereof.

In some embodiments, the coding sequence for CDKL5 comprises a nucleotide sequence selected from the group consisting of SEQ ID NOs: 12-18 and 19, or a nucleotide sequence at least 95%, at least 96%, at least 97%, at least 98%, at least 99%, at least 99.5%, or at least 99.9% identical to any of SEQ ID NOs: 12-19.

In an exemplary embodiment, the rAAV comprises an AAV capsid and a vector genome packaged therein, wherein the vector genome comprises the nucleotide sequence of SEQ ID NO: 20, or a nucleotide sequence at least 95%, at least 96%, at least 97%, at least 98%, at least 99%, at least 99.5%, or at least 99.9% identical thereto. In some such embodiments, the AAV capsid is an AAV9 capsid.

### Inverted Terminal Repeats (ITRs)

In some embodiments, the rAAV comprises a packaged vector genome which comprises an AAV ITR sequence, which functions as both the origin of vector DNA replication and the packaging signal of the vector genome, when AAV and adenovirus helper functions are provided in trans. Additionally, the ITRs serve as the target for single-stranded endonucleatic nicking by the large Rep proteins, resolving individual genomes from replication intermediates.

In some embodiments, the 5′-ITR sequence is from AAV2. In some embodiments, the 3′-ITR sequence is from AAV2. In some embodiments, the 5′-ITR sequence and the 3′-ITR sequence are from AAV2. In some embodiments, the 5′-ITR sequence and/or the 3′-ITR sequence are from AAV2 and comprise or consist of SEQ ID NO:2. In other embodiments, the 5′-ITR sequence and/or the 3′-ITR sequence are from a non-AAV2 source.

### Promoter

In various aspects described herein, the rAAV comprises a packaged vector genome which comprises a promoter sequence that helps drive and regulate expression of a heterologous gene. In exemplary embodiments, the promoter sequence is located between a 5′-ITR sequence and the partial or complete coding sequence for the heterologous gene. In some embodiments, the promoter sequence is located downstream of an enhancer sequence. In some embodiments the promoter sequence is located upstream of an intron sequence.

In some embodiments, the promoter is a neuron-specific promoter. In one embodiment, the neuron-specific promoter is selected from a human synapsin 1 (SYN1) promoter, a mouse calcium/calmodulin-dependent protein kinase II (CaMKII) promoter, a rat tubulin alpha I (Tal) promoter, a rat neuron-specific enolase (NSE) promoter, a human neuron-specific enolase (ENO2) promoter, a human platelet-derived growth factor-beta chain (PDGF) promoter, a human BM88 promoter, and a neuronal nicotinic receptor β2 (CHRNB2) promoter.

In an exemplary embodiment, the neuron-specific promoter is the SYN1 promoter (e.g., human SYN1 promoter). In one embodiment, the SYN1 promoter (e.g., human SYN1 promoter) is at least 80%, at least 85%, at least 90%, at least 91%, at least 92%, at least 93%, at least 94%, at least 95%, at least 96%, at least 97%, at least 98%, at least 99% or more identical to SEQ ID NO:3. In an exemplary embodiment, the SYN1 promoter (e.g., human SYN1 promoter) comprises or consists of SEQ ID NO:3.

In some embodiments, the promoter is selected from a chicken β-actin (CBA) promoter, a cytomegalovirus (CMV) immediate early gene promoter, a transthyretin (TTR) promoter, a thyroxine binding globulin (TBG) promoter, and an alpha-1 anti-trypsin (A1AT) promoter.

In an exemplary embodiment, the promoter is the CBA promoter. In one embodiment, the CBA promoter comprises or consists of SEQ ID NO:4.

In some embodiments, the promoter is a gene-specific endogenous promoter. In one embodiment, the promoter comprises native gene promoter elements.

### Other Packaged Vector Genome Elements

In addition to a promoter and a coding sequence for a heterologous gene, a packaged genome may contain other appropriate transcription initiation, termination, enhancer sequence, and efficient RNA processing signals. As described in further detail below, such sequences include splicing and polyadenylation (poly A) signals, regulatory elements that enhance expression, sequences that stabilize cytoplasmic mRNA, sequences that enhance translation efficiency (i.e., the Kozak consensus sequence), and sequences that enhance protein stability.

In some embodiments, the rAAV contains a packaged vector genome that comprises one or more enhancer sequences. In one embodiment, the enhancer is selected from a cytomegalovirus immediate early gene (CMV) enhancer, a transthyretin enhancer (enTTR), a chicken β-actin (CBA) enhancer, an En34 enhancer, and an ApoE enhancer. In an exemplary embodiment, the enhancer is the CMV enhancer (e.g., CMV immediate early gene enhancer). In one embodiment, the CMV enhancer (e.g., CMV immediate early gene enhancer) comprises or consists of SEQ ID NO:7.

In some embodiments, the rAAV contains a packaged vector genome that comprises one or more intron sequences. In one embodiment, the intron is selected from an SV40 Small T intron, a rabbit hemoglobin subunit beta (rHBB) intron, a human beta globin IVS2 intron, a β-globin/IgG chimeric intron, and an hFIX intron. In one exemplary embodiment, the intron is the SV40 Small T intron. In one embodiment, the SV40 Small T intron sequence comprises or consists of SEQ ID NO:8.

In some embodiments, the rAAV contains a packaged vector genome comprises a consensus Kozak sequence. In some embodiments, the consensus Kozak sequence is located downstream of an intron sequence. In one embodiment, the consensus Kozak sequence is GCCGCCACC.

In some embodiments, the rAAV contains a packaged vector genome that comprises a polyadenylation signal sequence. In one embodiment, the polyadenylation signal sequence is selected from a bovine growth hormone (BGH) polyadenylation signal sequence, an SV40 polyadenylation signal sequence, a rabbit beta globin polyadenylation signal sequence, and a gene-specific endogenous polyadenylation signal sequence. In an exemplary embodiment, the polyadenylation signal sequence is the SV40 polyadenylation signal sequence. In one embodiment, the SV40 polyadenylation signal sequence comprises or consists of SEQ ID NO:5.

II. Methods of Delivering rAAV

In one aspect, the present disclosure provides methods of delivering rAAV to the central nervous system of subject, comprising administering to the subject a pharmaceutical composition as described herein.

In an exemplary embodiment, the pharmaceutical composition is administered intrathecally, e.g., via intraventricular, lumbar, or intra-cisterna magna administration. In some embodiments, the pharmaceutical composition is administered by intra-cisterna magna administration.

For example, in some embodiments, the pharmaceutical composition is administered using an auto-intrathecal injector. For instance, an injector that leverages CSF dynamics, physiological pulsatility, and volume displacement can be utilized to deliver rAAV intrathecally. An example of one possible injector for use in the methods of the invention is the Pulsar™ Smart Intrathecal Delivery Platform in development at Alcyone Lifesciences, Inc.

The specific dose administered can be a uniform dose for each patient, for example, 1.0×10¹¹-1.0×10¹⁴ genome copies (GC) of virus per patient. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can also be adjusted as the progress of the disease is monitored

In some embodiments, the pharmaceutical composition is administered at an rAAV dose of, e.g., about 1.0×10¹¹ genome copies per kilogram of patient body weight (GC/kg) to about 1×10¹⁴ GC/kg, about 5×10¹¹ genome copies per kilogram of patient body weight (GC/kg) to about 5×10¹³ GC/kg, or about 1×10¹² to about 1×10¹³ GC/kg, as measured by qPCR or digital droplet PCR (ddPCR). In some embodiments, the rAAV is administered at a dose of about 1×10¹² to about 1× 10¹³ genome copies (GC)/kg. In some embodiments, the rAAV is administered at a dose of about 1.1×10¹¹, about 1.3×10¹¹, about 1.6×10¹¹, about 1.9×10¹¹, about 2×10¹¹, about 2.5×10¹¹, about 3.0×10¹¹, about 3.5×10¹¹, about 4.0×10¹¹, about 4.5×10¹¹, about 5.0×10¹¹, about 5.5×10¹¹, about 6.0×10¹¹, about 6.5×10¹¹, about 7.0×10¹¹, about 7.5×10¹¹, about 8.0×10¹¹, about 8.5×10¹¹, about 9.0×10¹¹, about 9.5×10¹¹, about 1.0×10¹², about 1.5×10¹², about 2.0×10¹², about 2.5×10¹², about 3.0×10¹², about 3.5×10¹², about 4.0×10¹², about 4.5×10¹², about 5.0×10¹², about 5.5×10¹², about 6.0×10¹², about 6.5×10¹², about 7.0×10¹², about 7.5×10¹², about 8.0×10¹², about 8.5×10¹², about 9.0×10¹², about 9.5×10¹², about 1.0×10¹³, about 1.5×10¹³, about 2.0×10¹³, about 2.5×10¹³, about 3.0×10¹³, about 3.5×10¹³, about 4.0×10¹³, about 4.5×10¹³, about 5.0×10¹³, about 5.5×10¹³, about 6.0×10¹³, about 6.5×10¹³, about 7.0×10¹³, about 7.5×10¹³, about 8.0×10¹³, about 8.5×10¹³, about 9.0×10¹³, about 9.5×10¹³ genome copies (GC)/kg. The rAAV can be administered in a single dose, or in multiple doses (such as 2, 3, 4, 5, 6, 7, 8, 9, 10 or more doses) as needed for the desired therapeutic results.

In some embodiments, the methods of delivering rAAV according to the instant invention may further comprise administration of an IgG-degrading protease prior to administration of a pharmaceutical composition described herein.

In some embodiments, the methods of delivering according to the instant invention is performed on a subject (e.g., a mammal, e.g., a human) who has been administered an IgG-degrading protease.

Examples of proteases that may be used in the instant invention include, for example and without limitation, those described in WO/2020/016318 and/or WO/2020/159970, including, for example, cysteine proteases from Streptococcus pyogenes, Streptococcus equi, Mycoplasma canis, Streptococcus agalactiae, Streptococcus pseudoporcinus, or Pseudomonas putida.

In certain embodiments, the IgG-degrading protease is the IdeS from Streptococcus pyogenes (SEQ ID NO:9) or a protease which is at least 80%, at least 85%, at least 90%, at least 95%, at least 96%, at least 97%, at least 98%, or at least 99% identical to SEQ ID NO:9. In some embodiments, the protease is an engineered variant of SEQ ID NO:9. Examples of engineered IdeS proteases are described in WO/2020/016318 and U.S. Patent Publication NOs. 20180023070 and 20180037962. In some embodiments, the engineered IdeS variant may have 1, 2, 3, 4, 5, or more amino acid modifications relative to SEQ ID NO:9.

In certain embodiments, the IgG-degrading protease is the IdeZ from Streptococcus equi (SEQ ID NO:10) or a protease which is at least 80%, at least 85%, at least 90%, at least 95%, at least 96%, at least 97%, at least 98%, or at least 99% identical to SEQ ID NO: 10. In some embodiments, the protease is an engineered variant of SEQ ID NO:10. Examples of engineered IdeZ proteases are described in WO/2020/016318. In some embodiments, the engineered IdeZ variant may have 1, 2, 3, 4, 5, or more amino acid modifications relative to SEQ ID NO:10.

Other proteases that may be used in the instant invention include, for example and without limitation, IgdE enzymes from Streptococcus suis, Streptococcus porcinus, and Streptococcus equi, described in WO/2017/134274.

In some embodiments, the IgG-degrading protease may be encapsulated in or complexed with liposomes, nanoparticles, lipid nanoparticles (LNPs), polymers, microparticles, microcapsules, micelles, or extracellular vesicles.

### Corticosteroids

In some embodiments, provided methods comprise administering a composition comprising rAAV as disclosed herein to a subject who has previously been administered a corticosteroid. The composition disclosed herein may be administered intrathecally, e.g., intracerebroventricularly, or via intra-cisterna magna delivery.

In various embodiments according to this aspect, the corticosteroid may be selected from prednisolone, prednisone, dexamethasone, hydrocortisone, triamcinolone, methylprednisolone, budesonide, betamethasone, and deflazacort. In an exemplary embodiment, the corticosteroid is prednisolone.

In various embodiments according to this aspect, the corticosteroid is administered to the subject at least about 12 hours before administration of the composition comprising rAAV. In another embodiment, the corticosteroid is administered to the subject at least about 24 hours before administration of the composition comprising rAAV. In yet another embodiment, the corticosteroid is administered to the subject at least about 2 days before administration of the composition comprising rAAV. In yet another embodiment, the corticosteroid is administered to the subject at least about 3, 4, 5, 6, 7, or more days before administration of the composition comprising rAAV. In yet another embodiment, the corticosteroid is administered to the subject at least about 7, 14, 21, or more days before administration of the composition comprising rAAV. In yet another embodiment, the corticosteroid is administered to the subject at least about 1 month, at least about 2 months, or at least about 3 months before administration of the composition comprising rAAV. In some embodiments, the corticosteroid is administered between 12 hours and 4 months, e.g., between 24 hours and 3 months before administration of the composition comprising rAAV.

In one embodiment, the corticosteroid is administered once before administration of the composition comprising rAAV. In another embodiment, the corticosteroid is administered twice before administration of the composition comprising rAAV. In yet another embodiment, the corticosteroid is administered 3, 4, 5, or more times before administration of the composition comprising rAAV.

Administration of the corticosteroid to a human subject can be by any route, including but not limited to oral, intravenous, intradermal, transdermal, subcutaneous, intramuscular, inhalation (e.g., via an aerosol), buccal (e.g., sub-lingual), topical (i.e., both skin and mucosal surfaces, including airway surfaces), intrathecal, intraarticular, intrapleural, intracerebral, intra-arterial, intraperitoneal, or intranasal administration. In an exemplary embodiment, the corticosteroid is administered orally.

In certain embodiments, the dose of a corticosteroid is measured in units of mg/kg of subject body weight. In other embodiments, the dose of a corticosteroid is measured in units of mg per dose administered to a subject. Any measurement of dose can be used in conjunction with compositions and methods of the invention and dosage units can be converted by means standard in the art.

In certain embodiments, the corticosteroid may be administered at a dose of about 1 mg to about 1000 mg. In some embodiments, the corticosteroid is administered at a dose of about 3 mg to about 300 mg. In some embodiments, the corticosteroid is administered at a dose of about 5 mg to about 150 mg. In some embodiments, the corticosteroid is administered at a dose of about 10 mg to about 100 mg. In some embodiments, the corticosteroid is administered at a dose of about 15 mg to about 80 mg. In some embodiments, the corticosteroid is administered at a dose of about 20 mg to about 60 mg.

In certain embodiments the corticosteroid may be administered at a dose of about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, or about 1000 mg.

In certain embodiments, the corticosteroid may be administered at a dose of about 0.1 mg/kg to about 100 mg/kg of body weight of a subject. In some embodiments, the anti-CD19 antibody is administered at a dose of about 0.2 mg/kg to about 10 mg/kg. In some embodiments, the anti-CD19 antibody is administered at a dose of about 0.5 mg/kg to about 5 mg/kg. In some embodiments, the anti-CD19 antibody is administered at a dose of about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 2 mg/kg, about 3 mg/kg, about 4 mg/kg, about 5 mg/kg, or about 10 mg/kg of body weight of a subject.

In some embodiments, the corticosteroid may be administered for a total of at least 1, at least 2, at least 3, at least 4, at least 5, at least 6, at least 7, at least 8, at least 9, at least 10, or more days prior to administration of the composition comprising rAAV. In some embodiments, the corticosteroid is administered between 1 day and 14 days, e.g., between 1 day and 12 days, or between 1 day and 10 days prior to the administration of the composition comprising rAAV. For example, in certain exemplary embodiments, the corticosteroid may be administered at 1 mg/kg per day for 5 days prior to administration of the composition comprising rAAV.

In some embodiments, the corticosteroid may be administered at 1 mg/kg per day for 4 weeks with a first dose occurring 5 days prior to administration of the composition comprising rAAV. In some embodiments, the corticosteroid may be administered at 1 mg/kg per day for 4 weeks with a first dose occurring 5 days prior to administration of the composition comprising rAAV, followed by a taper of corticosteroid for an additional 4 weeks.

The methods according to this aspect may be used to treat any CNS disorder for which gene therapy may be suitable. In some embodiments, the CNS disorder is selected from CDKL5-Deficiency Disorder (CDD), Angelman syndrome, Batten disease, Krabbe disease, Parkinson's disease, Alzheimer's disease, Spinal Muscular Atrophy (SMA) Types I, II, III, and IV, X-linked Myotubular Myopathy, Friedrich's Ataxia, Canavan's, Amyotrophic Lateral Sclerosis (ALS), Adrenoleukodystrophy, Huntington disease, Rett syndrome, and Spinocerebellar ataxia.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the present disclosure, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within the present disclosure, embodiments have been described and depicted in a way that enables a clear and concise disclosure to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including” is intended merely to illustrate better the present invention and does not pose a limitation on the scope of the invention unless claimed. No language in the specification should be construed as indicating any non-claimed element as essential to the practice of the present invention.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: Generation of rAAV Compositions

The present example describes various rAAV-containing compositions used in other Examples in the present disclosure.

rAAV expressing a heterologous gene were produced in human embryonic kidney (HEK) or HeLa cell producer lines. rAAV compositions were made using the following formulations.

TABLE 1
rAAV-containing formulations

| # | Buffer | Salt(s) | Polyhydric alcohol | Surfactant |
|---|---|---|---|---|
| 1 | 10 mM phosphate | 135 mM NaCl | 5% sorbitol | 0.001% Pluronic ® F68 |
| | | | (0.27M sorbitol) | (poloxamer 188) |
| 2 | 10 mM phosphate | 135 mM NaCl | 10% sorbitol | 0.001% Pluronic ® F68 |
| | | | (0.54M sorbitol) | (poloxamer 188) |
| 3 | 10 mM phosphate | 130 mM NaCl | 5% sorbitol | 0.001% Pluronic ® F68 |
| | | 2 mM KCl | (0.27M sorbitol) | (poloxamer 188) |
| | | 1 mM MgCl₂, | | |
| | | and | | |
| | | 1 mM CaCl₂ | | |

### Example 2: Biodistribution and Transduction Efficiency of rAAV-Containing Formulations

The present example demonstrates successful delivery of recombinant adeno-associated virus (rAAV) expressing a therapeutic gene product into various brain regions using compositions disclosed herein.

In the present example, rAAV expressing CDKL5 (AAV9-SYN-CDKL5) were generated and delivered to mice deficient in CDKL5.

CDKL5 deficiency disorder (CDD) is a rare neurodevelopmental disease caused by mutations in the CDKL5 gene which can manifest in a broad range of clinical symptoms and severity. The CDKL5 gene encodes a cyclin-dependent kinase-like 5 (CDKL5) protein that is essential for normal brain development and function. CDD is caused by pathogenic variants in the CDKL5 gene that include deletions, truncations, splice variants, and missense mutations. These variants can reduce the amount of functional CDKL5 protein and/or diminish its activity in neurons.

Compositions comprising HeLa-produced AAV9-SYN-CDKL5 and additional components as shown in Table 1 from Example 1 were made. CDKL5-deficient (Cdk15 KO) mice between 3 and 5 weeks of age were administered 10 μL of either a control or an rAAV composition as shown in Table 2, by intracerebroventricular (ICV) injection. The “control” groups were administered either saline (Group 2) or a control composition comprising HEK-produced AAV9-SYN-CDKL5 (Group 3). Wild type C57BL/6 mice were also used as a control (Group 1).

TABLE 2

| Group | Mice | n | Test article |
|---|---|---|---|
| 1 | C57BL/6 | 4 | Saline |
| 2 | CDKL5 KO | 4 | Saline |
| 3 | CDKL5 KO | 4 | Control composition |
| | | | (1.35 × 10¹² GC/mL HEK-produced AAV9-SYN-CDKL5) |
| 4 | CDKL5 KO | 4 | 2.8 × 10¹² GC/mL HeLa-produced AAV9-SYN-CDKL5 in 10 |
| | | | mM phosphate, 135 mM NaCl, 5% sorbitol, and 0.001% F68 |
| 5 | CDKL5 KO | 3 | 2.6 × 10¹² GC/mL HeLa-produced AAV9-SYN-CDKL5 in 10 |
| | | | mM phosphate, 135 mM NaCl, 10% sorbitol, and 0.001% |
| | | | F68 |
| 6 | CDKL5 KO | 3 | 3.5 × 10¹² GC/mL HeLa-produced AAV9-SYN-CDKL5 in |
| | | | 10 mM Tris, 130 mM NaCl, 2 mM KCl, 1 mM MgCl₂, |
| | | | 1 mM CaCl₂, 5% sorbitol, and 0.001% F68 |

Mice were sacrificed at 2 weeks after injection, and tissues were analyzed for presence of vector genomes (e.g., by quantitative polymerase chain reaction (qPCR)), vector RNA (e.g., using a BaseScope® assay), CDKL5 RNA (e.g., using an RNAScope® assay) or CDKL5 protein (e.g., by Western Blot).

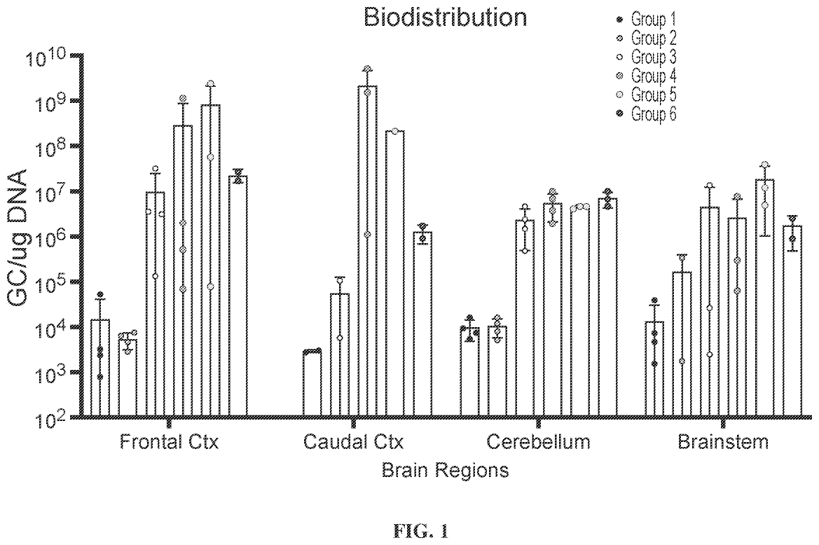

FIG. 1 shows the calculated number of genome copies (GC) per μg of DNA in various brain regions (frontal cortex, caudal cortex, cerebellum, and brainstem). For all three formulations tested (Groups 4, 5, and 6), high amounts (106 or greater GC/ug DNA) of vector genomes were observed in all brain regions assessed from brains of mice administered rAAV.

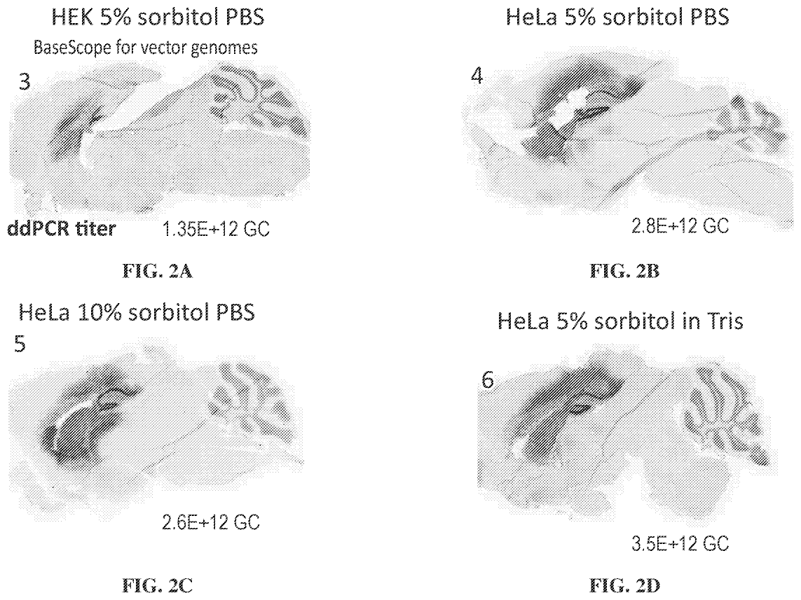

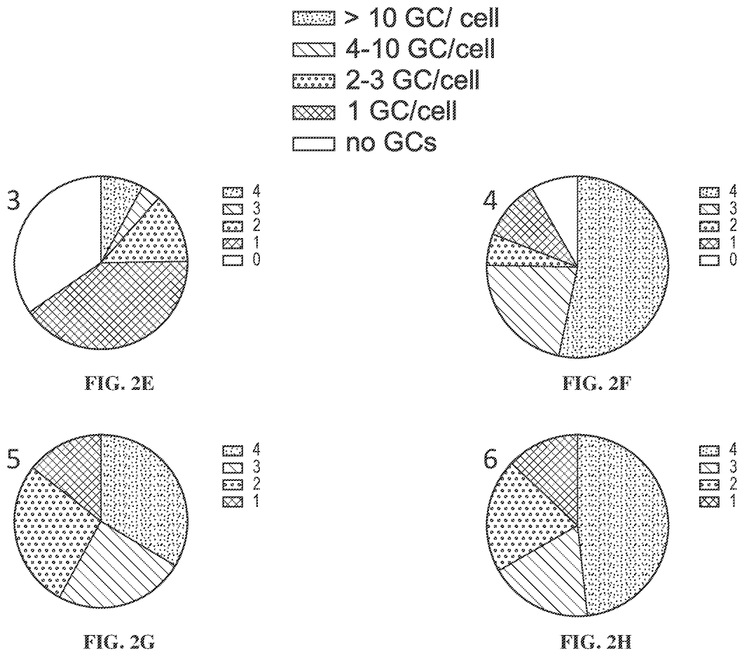

FIGS. 2A-2D depict representative images of tissue sections with staining corresponding to RNA expression of vector genomes (as detected by BaseScope®) in mice administered rAAV in groups 3 (FIG. 2A), 4 (FIG. 2B), 5 (FIG. 2C), and 6 (FIG. 2D). FIGS. 2E-2H are pie graphs depicting corresponding results from quantitation of BaseScope signals, indicative of transduction efficiency, in the cortex of mice administered control or rAAV formulations from Groups 3 (FIG. 2E), 4 (FIG. 2F), 5 (FIG. 2G), and 6 (FIG. 2H). Each pie graph is labeled on the left top corner with the number of the mouse group for the experiment. FIG. 2E shows the pie graph for mouse Group 3, which was administered with a control composition. FIGS. 2F, 2G, and 2H show the pie graphs for Groups 4, 5, and 6, respectively, which were each administered with rAAV formulations disclosed herein. The legends for each pie graph are on the right-hand side of each pie graph and indicate a number from 0 to 4 to represent the amount of genome copies (GC)/cell quantitated. The number 4 (fine stippling pattern) represents >10 GC/cell: the number 3 (stripes pattern) represents 4-10 GC/cell: the number 2 (medium stippling pattern) represents 2-3 GC/cell: the number 1 (cross-hatch pattern) represents 1 GC/cell; and the number 0 (solid white) represents no GC/cell. As shown in FIGS. 2F, 2G, and 2H (corresponding to results from Groups 4, 5, and 6, respectively), administration of rAAV using formulations disclosed herein resulted in delivery of >10 GC/cell in a significant percentage of cells in the cortex.

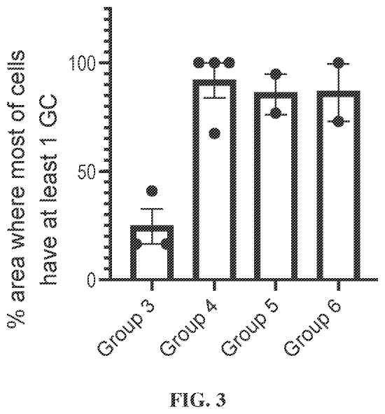

FIG. 3 is a bar graph that depicts the percentage area where most of the cells have at least 1 genome copy. In Groups 4, 5, and 6, this percentage was at least 80%.

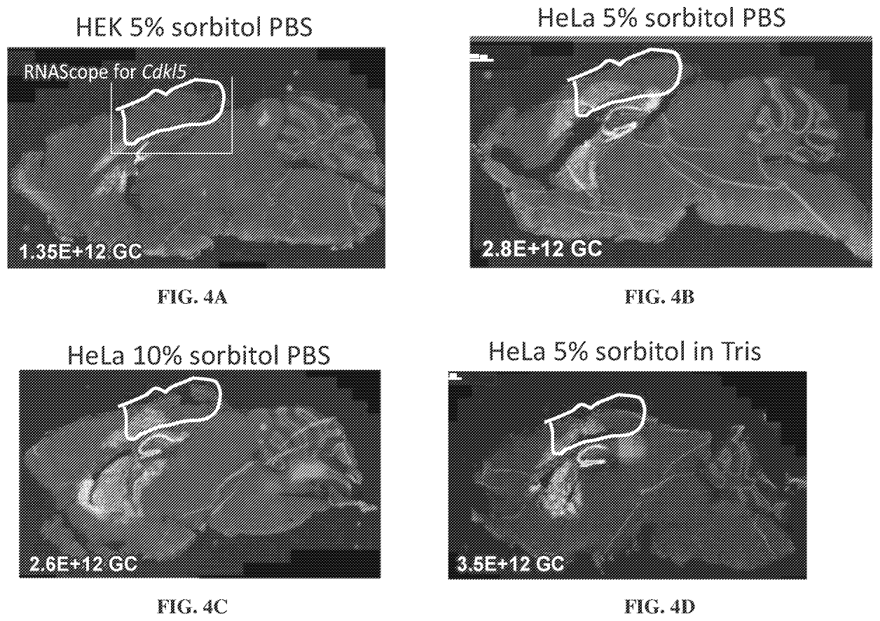

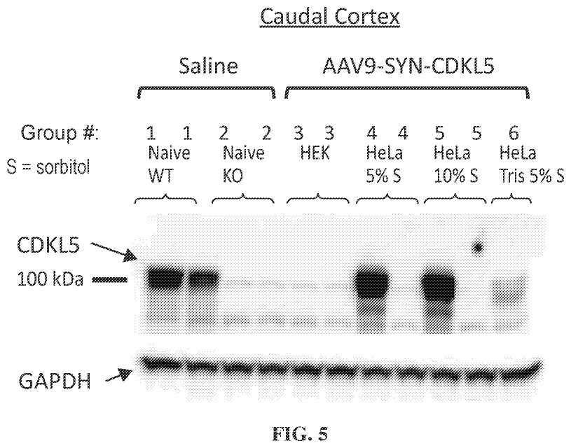

FIGS. 4A-4D depict representative images of tissue sections with staining corresponding to RNA expression of CDKL5 (as detected by RNAScope®) in mice administered rAAV in groups 3 (FIG. 4A), 4 (FIG. 4B), 5 (FIG. 4C), and 6 (FIG. 4D). FIG. 5 depicts a Western Blot stained for CDKL5 protein expression in caudal cortex tissues. Staining for GADPH protein expression is shown as a loading control.

Results described in this Example demonstrate that compositions disclosed herein can be used to deliver a recombinant rAAV to various brain tissues and express a heterologous gene in the brain.

### Example 3: Delivery of rAAV to the Central Nervous System by Intra-Cisterna Magna or Lumbar-Intrathecal Injection

This Example describes experiments that may be used to assess rAAV delivery to the central nervous system using formulations described herein, administered via the intra-cisterna magna or lumbar-intrathecal routes.

Formulations comprising an rAAV vector encoding a reporter gene (e.g., GFP) may be generated as described in Example 1. Non-human primates (e.g., cynomolgus monkeys) may be administered a volume of a formulation by injection into the cisterna magna or by lumbar-intrathecal injection. During administration, the non-human primate may be in the Trendelenburg position.

Biodistribution of the rAAV may be assessed, e.g., by the analyzing the amount of genome copies (GC per μg DNA) in various tissues. Delivery to the central nervous system may be assessed by analyzing GC per μg DNA in various brain tissues. Substantial amounts of genome copies across various brain tissues would indicate successful delivery to the central nervous system.

Expression of the reporter gene in various brain tissues and/or at the cellular level can be assessed, e.g., in tissue sections using staining or other visualization methods appropriate for the reporter gene.

### Example 4: Delivery of a Therapeutic rAAV Via Intra-Cisterna Magna Injection

An rAAV expressing a gene that encodes a protein associated with the central nervous system disorder may be generated. A composition comprising the rAAV may be formulated similarly as described in Example 1. The composition may be delivered to a subject in need thereof (e.g., subject who has been diagnosed with the central nervous system disorder or identified as at risk of developing the central nervous system disorder) via intra-cisterna magna injection. Treatment, amelioration, or prevention outcomes may be measured in the subject by methods appropriate for the relevant central nervous system disorder.

### Example 5: Delivery of a Therapeutic rAAV Via Intra Lumbar-IT Injection

An rAAV expressing a gene that encodes a protein associated with the central nervous system disorder may be generated. A composition comprising the rAAV may be formulated similarly as described in Example 1. The composition may be delivered to a subject in need thereof (e.g., a subject who has been diagnosed with the central nervous system disorder or identified as at risk of developing the central nervous system disorder) via lumbar-IT injection. Treatment, amelioration, or prevention outcomes may be measured in the subject by methods appropriate for the relevant central nervous system disorder.

### Example 6: Delivery of a Therapeutic rAAV Via Intracerebroventricular Injection

An rAAV expressing a gene that encodes a protein associated with the central nervous system disorder may be generated. A composition comprising the rAAV may be formulated similarly as described in Example 1. The composition may be delivered to a subject in need thereof (e.g., a subject who has been diagnosed with the central nervous system disorder or identified as at risk of developing the central nervous system disorder) via intracerebroventricular injection. Treatment, amelioration, or prevention outcomes may be measured in the subject by methods appropriate for the relevant central nervous system disorder.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

SEQ ID NO: 1 (AAV9 amino acid sequence)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
KTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQAQAQTGWVQNQGILPGMVWQDRDVYLQGPIWAKIPHTDGNFHPSPLMGGFGM
KHPPPQILIKNTPVPADPPTAFNKDKLNSFITQYSTGQVSVEIEWELQKENSKRWNPEIQYTSN
YYKSNNVEFAVNTEGVYSEPRPIGTRYLTRNL
SEQ ID NO: 2 (AAV2 ITR)
TTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGAGAGGGAGTGGCCAACTC
CATCACTAGGGGTTCCT
SEQ ID NO: 3 (SYN1 promoter)
AGTGCAAGTGGGTTTTAGGACCAGGATGAGGCGGGGTGGGGGTGCCTACCTGACGACCGACCCC
GACCCACTGGACAAGCACCCAACCCCCATTCCCCAAATTGCGCATCCCCTATCAGAGAGGGGGA
GGGGAAACAGGATGCGGCGAGGCGCGTGCGCACTGCCAGCTTCAGCACCGCGGACAGTGCCTTC
GCCCCCGCCTGGCGGCGCGCGCCACCGCCGCCTCAGCACTGAAGGCGCGCTGACGTCACTCGCC
GGTCCCCCGCAAACTCCCCTTCCCGGCCACCTTGGTCGCGTCCGCGCCGCCGCCGGCCCAGCCG
GACCGCACCACGCGAGGCGCGAGATAGGGGGGCACGGGCGCGACCATCTGCGCTGCGGCGCCGG
CGACTCAGCGCTGCCTCAGTCTGCGGTGGGCAGCGGAGGAGTCGTGTCGTGCCTGAGAGCGCAG
SEQ ID NO: 4 (CBA promoter)
TCGAGGTGAGCCCCACGTTCTGCTTCACTCTCCCCATCTCCCCCCCCTCCCCACCCCCAATTTT
GTATTTATTTATTTTTTAATTATTTTATGCAGCGATGGGGGCGGGGGGGGGGGGGGCGCGCGCC
AGGCGGGGCGGGGCGGGGCGAGGGGGGGGCGGGGCGAGGCGGAGAGGTGCGGCGGCAGCCAAT
CAGAGCGGCGCGCTCCGAAAGTTTCCTTTTATGGCGAGGCGGCGGCGGCGGCGGCCCTATAAAA
AGCGAAGCGCGCGGCGGGCG
SEQ ID NO: 5 (SV40 Polyadenylation Signal)
GATCCAGACATGATAAGATACATTGATGAGTTTGGACAAACCACAACTAGAATGCAGTGAAAAA
AATGCTTTATTTGTGAAATTTGTGATGCTATTGCTTTATTTGTAACCATTATAAGCTGCAATAA
ACAAGTTAACAACAACAATTGCATTCATTTTATGTTTCAGGTTCAGGGGGAGGTGTGGGAGGTT
TTTTAG
(Consensus Kozak Sequence)
GCCGCCACC
SEQ ID NO: 7 (CMV Enhancer)
CGTTACATAACTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGTCAATGGGTGG
ACTATTTACGGTAAACTGCCCACTTGGCAGTACATCAAGTGTATCATATGCCAAGTACGCCCCC
TATTGACGTCAATGACGGTAAATGGCCCGCCTGGCATTATGCCCAGTACATGACCTTATGGGAC
TTTCCTACTTGGCAGTACATCTACGTATTAGTCATCGCTATTACCATG
SEQ ID NO: 8 (SV40 Intron)
GCTCTAAGGTAAATATAAAATTTTTAAGTGTATAATGTGTTAAACTACTGATTCTAATTGTTTC
TCTCTTTTAGATTCCAACCTTTGGAACTGAT
SEQ ID NO: 9 (Streptococcus pyogenes IdeS)
DSFSANQEIRYSEVTPYHVTSVWTKGVTPPANFTQGEDVFHAPYVANQGWYDITKTFNGKDDLL
CGAATAGNMLHWWFDQNKDQIKRYLEEHPEKQKINFNGEQMFDVKEAIDTKNHQLDSKLFEYFK
EKAFPYLSTKHLGVFPDHVIDMFINGYRLSLTNHGPTPVKEGSKDPRGGIFDAVFTRGDQSKLL
TSRHDFKEKNLKEISDLIKKELTEGKALGLSHTYANVRINHVINLWGADFDSNGNLKAIYVTDS
DSNASIGMKKYFVGVNSAGKVAISAKEIKEDNIGAQVLGLFTLSTGQDSWNQTN
SEQ ID NO: 10 (Streptococcus equi IdeZ)
MKTIAYPNKPHSLSAGLLTAIAIFSLASSNITYADDYQRNATEAYAKEVPHQITSVWSKGVTPL
TPEQFRYNNEDVIHAPYLAHQGWYDITKAFDGKDNLLCGAATAGNMLHWWFDQNKTEIEAYLSK
HPEKQKIIFNNQELFDLKAAIDTKDSQTNSQLFNYFRDKAFPNLSARQLGVMPDLVLDMFINGY
YLNVFKTQSTDVNRPYQDKDKRGGIFDAVFTRGDQTTLLTARHDLKNKGLNDISTIIKQELTEG
RALALSHTYANVSISHVINLWGADFNAEGNLEAIYVTDSDANASIGMKKYFVGINAHGHVAISA
KKIEGENIGAQVLGLFTLSSGKDIWQKLS
SEQ ID NO: 11 (AAV9 Nucleic Acid Sequence)
ATGGCTGCCGATGGTTATCTTCCAGATTGGCTCGAGGACAACCTTAGTGAAGGAATTCGCGAGT
GGTGGGCTTTGAAACCTGGAGCCCCTCAACCCAAGGCAAATCAACAACATCAAGACAACGCTCG
AGGTCTTGTGCTTCCGGGTTACAAATACCTTGGACCCGGCAACGGACTCGACAAGGGGGAGCCG
GTCAACGCAGCAGACGCGGCGGCCCTCGAGCACGACAAGGCCTACGACCAGCAGCTCAAGGCCG
GAGACAACCCGTACCTCAAGTACAACCACGCCGACGCCGAGTTCCAGGAGCGGCTCAAAGAAGA
TACGTCTTTTGGGGGCAACCTCGGGCGAGCAGTCTTCCAGGCCAAAAAGAGGCTTCTTGAACCT
CTTGGTCTGGTTGAGGAAGCGGCTAAGACGGCTCCTGGAAAGAAGAGGCCTGTAGAGCAGTCTC
CTCAGGAACCGGACTCCTCCGCGGGTATTGGCAAATCGGGTGCACAGCCCGCTAAAAAGAGACT
CAATTTCGGTCAGACTGGCGACACAGAGTCAGTCCCAGACCCTCAACCAATCGGAGAACCTCCC
GCAGCCCCCTCAGGTGTGGGATCTCTTACAATGGCTTCAGGTGGTGGCGCACCAGTGGCAGACA
ATAACGAAGGTGCCGATGGAGTGGGTAGTTCCTCGGGAAATTGGCATTGCGATTCCCAATGGCT
GGGGGACAGAGTCATCACCACCAGCACCCGAACCTGGGCCCTGCCCACCTACAACAATCACCTC
TACAAGCAAATCTCCAACAGCACATCTGGAGGATCTTCAAATGACAACGCCTACTTCGGCTACA
GCACCCCCTGGGGGTATTTTGACTTCAACAGATTCCACTGCCACTTCTCACCACGTGACTGGCA
GCGACTCATCAACAACAACTGGGGATTCCGGCCTAAGCGACTCAACTTCAAGCTCTTCAACATT
CAGGTCAAAGAGGTTACGGACAACAATGGAGTCAAGACCATCGCCAATAACCTTACCAGCACGG
TCCAGGTCTTCACGGACTCAGACTATCAGCTCCCGTACGTGCTCGGGTCGGCTCACGAGGGCTG
CCTCCCGCCGTTCCCAGCGGACGTTTTCATGATTCCTCAGTACGGGTATCTGACGCTTAATGAT
GGAAGCCAGGCCGTGGGTCGTTCGTCCTTTTACTGCCTGGAATATTTCCCGTCGCAAATGCTAA
GAACGGGTAACAACTTCCAGTTCAGCTACGAGTTTGAGAACGTACCTTTCCATAGCAGCTACGC
TCACAGCCAAAGCCTGGACCGACTAATGAATCCACTCATCGACCAATACTTGTACTATCTCTCA
AAGACTATTAACGGTTCTGGACAGAATCAACAAACGCTAAAATTCAGTGTGGCCGGACCCAGCA
ACATGGCTGTCCAGGGAAGAAACTACATACCTGGACCCAGCTACCGACAACAACGTGTCTCAAC
CACTGTGACTCAAAACAACAACAGCGAATTTGCTTGGCCTGGAGCTTCTTCTTGGGCTCTCAAT
GGACGTAATAGCTTGATGAATCCTGGACCTGCTATGGCCAGCCACAAAGAAGGAGAGGACCGTT
TCTTTCCTTTGTCTGGATCTTTAATTTTTGGCAAACAAGGAACTGGAAGAGACAACGTGGATGC
GGACAAAGTCATGATAACCAACGAAGAAGAAATTAAAACTACTAACCCGGTAGCAACGGAGTCC
TATGGACAAGTGGCCACAAACCACCAGAGTGCCCAAGCACAGGCGCAGACCGGCTGGGTTCAAA
ACCAAGGAATACTTCCGGGTATGGTTTGGCAGGACAGAGATGTGTACCTGCAAGGACCCATTTG
GGCCAAAATTCCTCACACGGACGGCAACTTTCACCCTTCTCCGCTGATGGGAGGGTTTGGAATG
AAGCACCCGCCTCCTCAGATCCTCATCAAAAACACACCTGTACCTGCGGATCCTCCAACGGCCT
TCAACAAGGACAAGCTGAACTCTTTCATCACCCAGTATTCTACTGGCCAAGTCAGCGTGGAGAT
CGAGTGGGAGCTGCAGAAGGAAAACAGCAAGCGCTGGAACCCGGAGATCCAGTACACTTCCAAC
TATTACAAGTCTAATAATGTTGAATTTGCTGTTAATACTGAAGGTGTATATAGTGAACCCCGCC
CCATTGGCACCAGATACCTGACTCGTAATCTGTAA
SEQ ID NO: 12 (CDKL5 Brain Isoform-Isoform 2)
ATGAAGATTCCTAACATTGGTAATGTGATGAATAAATTTGAGATCCTTGGGGTTGTAGGTGAAG
GAGCCTATGGAGTTGTACTTAAATGCAGACACAAGGAAACACATGAAATTGTGGCGATCAAGAA
ATTCAAGGACAGTGAAGAAAATGAAGAAGTCAAAGAAACGACTTTACGAGAGCTTAAAATGCTT
CGGACTCTCAAGCAGGAAAACATTGTGGAGTTGAAGGAAGCATTTCGTCGGAGGGGAAAGTTGT
ACTTGGTGTTTGAGTATGTTGAAAAAAATATGCTCGAATTGCTGGAAGAAATGCCAAATGGAGT
TCCACCTGAGAAAGTAAAAAGCTACATCTATCAGCTAATCAAGGCTATTCACTGGTGCCATAAG
AATGATATTGTCCATCGAGATATAAAACCAGAAAATCTCTTAATCAGCCACAATGATGTCCTAA
AACTGTGTGACTTTGGTTTTGCTCGTAATCTGTCAGAAGGCAATAATGCTAATTACACAGAGTA
CGTTGCCACCAGATGGTATCGGTCCCCAGAACTCTTACTTGGCGCTCCCTATGGAAAGTCCGTG
GACATGTGGTCGGTGGGCTGTATTCTTGGGGAGCTTAGCGATGGACAGCCTTTATTTCCTGGAG
AAAGTGAAATTGACCAACTTTTTACTATTCAGAAGGTGCTAGGACCACTTCCATCTGAGCAGAT
GAAGCTTTTCTACAGTAATCCTCGCTTCCATGGGCTCCGGTTTCCAGCTGTTAACCATCCTCAG
TCCTTGGAAAGAAGATACCTTGGAATTTTGAATAGTGTTCTACTTGACCTAATGAAGAATTTAC
TGAAGTTGGACCCAGCTGACAGATACTTGACAGAACAGTGTTTGAATCACCCTACATTTCAAAC
CCAGAGACTTCTGGATCGTTCTCCTTCAAGGTCAGCAAAAAGAAAACCTTACCATGTGGAAAGC
AGCACATTGTCTAATAGAAACCAAGCCGGCAAAAGTACTGCTTTGCAGTCTCACCACAGATCTA
ACAGCAAGGACATCCAGAACCTGAGTGTAGGCCTGCCCCGGGCTGACGAAGGTCTCCCTGCCAA
TGAAAGCTTCCTAAATGGAAACCTTGCTGGAGCTAGTCTTAGTCCACTGCACACCAAAACCTAC
CAAGCAAGCAGCCAGCCTGGGTCTACCAGCAAAGATCTCACCAACAACAACATACCACACCTTC
TTAGCCCAAAAGAAGCCAAGTCAAAAACAGAGTTTGATTTTAATATTGACCCAAAGCCTTCAGA
AGGCCCAGGGACAAAGTACCTCAAGTCAAACAGCAGATCTCAGCAGAACCGCCACTCATTCATG
GAAAGCTCTCAAAGCAAAGCTGGGACACTGCAGCCCAATGAAAAGCAGAGTCGGCATAGCTATA
TTGACACAATTCCCCAGTCCTCTAGGAGTCCCTCCTACAGGACCAAGGCCAAAAGCCATGGGGC
ACTGAGTGACTCCAAGTCTGTGAGCAACCTTTCTGAAGCCAGGGCCCAAATTGCGGAGCCCAGT
ACCAGTAGGTACTTCCCATCTAGCTGCTTAGACTTGAATTCTCCCACCAGCCCAACCCCCACCA
GACACAGTGACACGAGAACTTTGCTCAGCCCTTCTGGAAGAAATAACCGAAATGAGGGAACGCT
GGACTCACGTCGAACCACAACCAGACATTCTAAGACGATGGAGGAATTGAAGCTGCCGGAGCAC
ATGGACAGTAGCCATTCCCATTCACTGTCTGCACCTCACGAATCTTTTTCTTATGGACTGGGCT
ACACCAGCCCCTTTTCTTCCCAGCAACGTCCTCATAGGCATTCTATGTATGTGACCCGTGACAA
AGTGAGAGCCAAGGGCTTGGATGGAAGCTTGAGCATAGGGCAAGGGATGGCAGCTAGAGCCAAC
AGCCTGCAACTCTTGTCACCCCAGCCTGGAGAACAGCTCCCTCCAGAGATGACTGTGGCAAGAT
CTTCGGTCAAAGAGACCTCCAGAGAAGGCACCTCTTCCTTCCATACACGCCAGAAGTCTGAGGG
TGGAGTGTATCATGACCCACACTCTGATGATGGCACAGCCCCCAAAGAAAATAGACACCTATAC
AATGATCCTGTGCCAAGGAGAGTTGGTAGCTTTTACAGAGTGCCATCTCCACGTCCAGACAATT
CTTTCCATGAAAATAATGTGTCAACTAGAGTTTCTTCTCTACCATCAGAGAGCAGTTCTGGAAC
CAACCACTCAAAAAGACAACCAGCATTCGATCCATGGAAAAGTCCTGAAAATATTAGTCATTCA
GAGCAACTCAAGGAAAAAGAGAAGCAAGGATTTTTCAGGTCAATGAAAAAGAAAAAGAAGAAAT
CTCAAACAGTACCCAATTCCGACAGCCCTGATCTTCTGACGTTGCAGAAATCCATTCATTCTGC
TAGCACTCCAAGCAGCAGACCAAAGGAGTGGCGCCCCGAGAAGATCTCAGATCTGCAGACCCAA
AGCCAGCCATTAAAATCACTGCGCAAGTTGTTACATCTCTCTTCGGCCTCAAATCACCCGGCTT
CCTCAGATCCCCGCTTCCAGCCCTTAACAGCTCAACAAACCAAAAATTCCTTCTCAGAAATTCG
GATTCACCCCCTGAGCCAGGCCTCTGGCGGGAGCAGCAACATCCGGCAGGAACCCGCACCGAAG
GGCAGGCCAGCCCTCCAGCTGCCAGGTCAGATGGATCCTGGTTGGCATGTGTCCTCTGTGACCA
GGAGTGCCACAGAGGGCCCTTCCTACTCTGAACAGCTGGGTGCCAAAAGTGGGCCAAATGGGCA
CCCCTATAACAGAACAAATCGCTCACGAATGCCAAATCTGAATGATTTAAAAGAGACAGCCTTG
SEQ ID NO: 13 (CDKL5 Canonical Isoform-Isoform 1)
ATGAAGATTCCTAACATTGGTAATGTGATGAATAAATTTGAGATCCTTGGGGTTGTAGGTGAAG
GAGCCTATGGAGTTGTACTTAAATGCAGACACAAGGAAACACATGAAATTGTGGCGATCAAGAA
ATTCAAGGACAGTGAAGAAAATGAAGAAGTCAAAGAAACGACTTTACGAGAGCTTAAAATGCTT
CGGACTCTCAAGCAGGAAAACATTGTGGAGTTGAAGGAAGCATTTCGTCGGAGGGGAAAGTTGT
ACTTGGTGTTTGAGTATGTTGAAAAAAATATGCTCGAATTGCTGGAAGAAATGCCAAATGGAGT
TCCACCTGAGAAAGTAAAAAGCTACATCTATCAGCTAATCAAGGCTATTCACTGGTGCCATAAG
AATGATATTGTCCATCGAGATATAAAACCAGAAAATCTCTTAATCAGCCACAATGATGTCCTAA
AACTGTGTGACTTTGGTTTTGCTCGTAATCTGTCAGAAGGCAATAATGCTAATTACACAGAGTA
CGTTGCCACCAGATGGTATCGGTCCCCAGAACTCTTACTTGGCGCTCCCTATGGAAAGTCCGTG
GACATGTGGTCGGTGGGCTGTATTCTTGGGGAGCTTAGCGATGGACAGCCTTTATTTCCTGGAG
AAAGTGAAATTGACCAACTTTTTACTATTCAGAAGGTGCTAGGACCACTTCCATCTGAGCAGAT
GAAGCTTTTCTACAGTAATCCTCGCTTCCATGGGCTCCGGTTTCCAGCTGTTAACCATCCTCAG
TCCTTGGAAAGAAGATACCTTGGAATTTTGAATAGTGTTCTACTTGACCTAATGAAGAATTTAC
TGAAGTTGGACCCAGCTGACAGATACTTGACAGAACAGTGTTTGAATCACCCTACATTTCAAAC
CCAGAGACTTCTGGATCGTTCTCCTTCAAGGTCAGCAAAAAGAAAACCTTACCATGTGGAAAGC
AGCACATTGTCTAATAGAAACCAAGCCGGCAAAAGTACTGCTTTGCAGTCTCACCACAGATCTA
ACAGCAAGGACATCCAGAACCTGAGTGTAGGCCTGCCCCGGGCTGACGAAGGTCTCCCTGCCAA
TGAAAGCTTCCTAAATGGAAACCTTGCTGGAGCTAGTCTTAGTCCACTGCACACCAAAACCTAC
CAAGCAAGCAGCCAGCCTGGGTCTACCAGCAAAGATCTCACCAACAACAACATACCACACCTTC
TTAGCCCAAAAGAAGCCAAGTCAAAAACAGAGTTTGATTTTAATATTGACCCAAAGCCTTCAGA
AGGCCCAGGGACAAAGTACCTCAAGTCAAACAGCAGATCTCAGCAGAACCGCCACTCATTCATG
GAAAGCTCTCAAAGCAAAGCTGGGACACTGCAGCCCAATGAAAAGCAGAGTCGGCATAGCTATA
TTGACACAATTCCCCAGTCCTCTAGGAGTCCCTCCTACAGGACCAAGGCCAAAAGCCATGGGGC
ACTGAGTGACTCCAAGTCTGTGAGCAACCTTTCTGAAGCCAGGGCCCAAATTGCGGAGCCCAGT
ACCAGTAGGTACTTCCCATCTAGCTGCTTAGACTTGAATTCTCCCACCAGCCCAACCCCCACCA
GACACAGTGACACGAGAACTTTGCTCAGCCCTTCTGGAAGAAATAACCGAAATGAGGGAACGCT
GGACTCACGTCGAACCACAACCAGACATTCTAAGACGATGGAGGAATTGAAGCTGCCGGAGCAC
ATGGACAGTAGCCATTCCCATTCACTGTCTGCACCTCACGAATCTTTTTCTTATGGACTGGGCT
ACACCAGCCCCTTTTCTTCCCAGCAACGTCCTCATAGGCATTCTATGTATGTGACCCGTGACAA
AGTGAGAGCCAAGGGCTTGGATGGAAGCTTGAGCATAGGGCAAGGGATGGCAGCTAGAGCCAAC
AGCCTGCAACTCTTGTCACCCCAGCCTGGAGAACAGCTCCCTCCAGAGATGACTGTGGCAAGAT
CTTCGGTCAAAGAGACCTCCAGAGAAGGCACCTCTTCCTTCCATACACGCCAGAAGTCTGAGGG
TGGAGTGTATCATGACCCACACTCTGATGATGGCACAGCCCCCAAAGAAAATAGACACCTATAC
AATGATCCTGTGCCAAGGAGAGTTGGTAGCTTTTACAGAGTGCCATCTCCACGTCCAGACAATT
CTTTCCATGAAAATAATGTGTCAACTAGAGTTTCTTCTCTACCATCAGAGAGCAGTTCTGGAAC
CAACCACTCAAAAAGACAACCAGCATTCGATCCATGGAAAAGTCCTGAAAATATTAGTCATTCA
GAGCAACTCAAGGAAAAAGAGAAGCAAGGATTTTTCAGGTCAATGAAAAAGAAAAAGAAGAAAT
CTCAAACAGTACCCAATTCCGACAGCCCTGATCTTCTGACGTTGCAGAAATCCATTCATTCTGC
TAGCACTCCAAGCAGCAGACCAAAGGAGTGGCGCCCCGAGAAGATCTCAGATCTGCAGACCCAA
AGCCAGCCATTAAAATCACTGCGCAAGTTGTTACATCTCTCTTCGGCCTCAAATCACCCGGCTT
CCTCAGATCCCCGCTTCCAGCCCTTAACAGCTCAACAAACCAAAAATTCCTTCTCAGAAATTCG
GATTCACCCCCTGAGCCAGGCCTCTGGCGGGAGCAGCAACATCCGGCAGGAACCCGCACCGAAG
GGCAGGCCAGCCCTCCAGCTGCCAGACGGTGGATGTGATGGCAGAAGACAGAGACACCATTCTG
GACCCCAAGATAGACGCTTCATGTTAAGGACGACAGAACAACAAGGAGAATACTTCTGCTGTGG
TGACCCAAAGAAGCCTCACACTCCGTGCGTCCCAAACCGAGCCCTTCATCGTCCAATCTCCAGT
CCTGCTCCCTATCCAGTACTCCAGGTCCGAGGCACTTCCATGTGCCCGACACTCCAGGTCCGAG
GCACTGATGCTTTCAGCTGCCCAACCCAGCAATCCGGGTTCTCTTTCTTCGTGAGACACGTTAT
GAGGGAAGCCCTGATTCACAGGGCCCAGGTAAACCAAGCTGCGCTCCTGACATACCATGAGAAT
GCGGCACTGACGGGCAAG
SEQ ID NO: 14 (CDKL5 Brain Isoform-Isoform 2-codon-opt 1)
ATGAAGATTCCTAATATTGGGAATGTGATGAATAAGTTTGAGATTCTGGGGGTGGTGGGGGAGG
GGGCTTATGGGGTGGTGCTGAAGTGTAGGCATAAGGAGACACATGAGATTGTGGCTATTAAGAA
GTTTAAGGATTCTGAGGAGAATGAGGAGGTGAAGGAGACAACACTGAGGGAGCTGAAGATGCTG
AGGACACTGAAGCAGGAGAATATTGTGGAGCTGAAGGAGGCTTTTAGGAGGAGGGGGAAGCTGT
ATCTGGTGTTTGAGTATGTGGAGAAGAATATGCTGGAGCTGCTGGAGGAGATGCCTAATGGGGT
GCCTCCTGAGAAGGTGAAGTCTTATATTTATCAGCTGATTAAGGCTATTCATTGGTGTCATAAG
AATGATATTGTGCATAGGGATATTAAGCCTGAGAATCTGCTGATTTCTCATAATGATGTGCTGA
AGCTGTGTGATTTTGGGTTTGCTAGGAATCTGTCTGAGGGGAATAATGCTAATTATACAGAGTA
TGTGGCTACAAGGTGGTATAGGTCTCCTGAGCTGCTGCTGGGGGCTCCTTATGGGAAGTCTGTG
GATATGTGGTCTGTGGGGTGTATTCTGGGGGAGCTGTCTGATGGGCAGCCTCTGTTTCCTGGGG
AGTCTGAGATTGATCAGCTGTTTACAATTCAGAAGGTGCTGGGGCCTCTGCCTTCTGAGCAGAT
GAAGCTGTTTTATTCTAATCCTAGGTTTCATGGGCTGAGGTTTCCTGCTGTGAATCATCCTCAG
TCTCTGGAGAGGAGGTATCTGGGGATTCTGAATTCTGTGCTGCTGGATCTGATGAAGAATCTGC
TGAAGCTGGATCCTGCTGATAGGTATCTGACAGAGCAGTGTCTGAATCATCCTACATTTCAGAC
ACAGAGGCTGCTGGATAGGTCTCCTTCTAGGTCTGCTAAGAGGAAGCCTTATCATGTGGAGTCT
TCTACACTGTCTAATAGGAATCAGGCTGGGAAGTCTACAGCTCTGCAGTCTCATCATAGGTCTA
ATTCTAAGGATATTCAGAATCTGTCTGTGGGGCTGCCTAGGGCTGATGAGGGGCTGCCTGCTAA
TGAGTCTTTTCTGAATGGGAATCTGGCTGGGGCTTCTCTGTCTCCTCTGCATACAAAGACATAT
CAGGCTTCTTCTCAGCCTGGGTCTACATCTAAGGATCTGACAAATAATAATATTCCTCATCTGC
TGTCTCCTAAGGAGGCTAAGTCTAAGACAGAGTTTGATTTTAATATTGATCCTAAGCCTTCTGA
GGGGCCTGGGACAAAGTATCTGAAGTCTAATTCTAGGTCTCAGCAGAATAGGCATTCTTTTATG
GAGTCTTCTCAGTCTAAGGCTGGGACACTGCAGCCTAATGAGAAGCAGTCTAGGCATTCTTATA
TTGATACAATTCCTCAGTCTTCTAGGTCTCCTTCTTATAGGACAAAGGCTAAGTCTCATGGGGC
TCTGTCTGATTCTAAGTCTGTGTCTAATCTGTCTGAGGCTAGGGCTCAGATTGCTGAGCCTTCT
ACATCTAGGTATTTTCCTTCTTCTTGTCTGGATCTGAATTCTCCTACATCTCCTACACCTACAA
GGCATTCTGATACAAGGACACTGCTGTCTCCTTCTGGGAGGAATAATAGGAATGAGGGGACACT
GGATTCTAGGAGGACAACAACAAGGCATTCTAAGACAATGGAGGAGCTGAAGCTGCCTGAGCAT
ATGGATTCTTCTCATTCTCATTCTCTGTCTGCTCCTCATGAGTCTTTTTCTTATGGGCTGGGGT
ATACATCTCCTTTTTCTTCTCAGCAGAGGCCTCATAGGCATTCTATGTATGTGACAAGGGATAA
GGTGAGGGCTAAGGGGCTGGATGGGTCTCTGTCTATTGGGCAGGGGATGGCTGCTAGGGCTAAT
TCTCTGCAGCTGCTGTCTCCTCAGCCTGGGGAGCAGCTGCCTCCTGAGATGACAGTGGCTAGGT
CTTCTGTGAAGGAGACATCTAGGGAGGGGACATCTTCTTTTCATACAAGGCAGAAGTCTGAGGG
GGGGGTGTATCATGATCCTCATTCTGATGATGGGACAGCTCCTAAGGAGAATAGGCATCTGTAT
AATGATCCTGTGCCTAGGAGGGTGGGGTCTTTTTATAGGGTGCCTTCTCCTAGGCCTGATAATT
CTTTTCATGAGAATAATGTGTCTACAAGGGTGTCTTCTCTGCCTTCTGAGTCTTCTTCTGGGAC
AAATCATTCTAAGAGGCAGCCTGCTTTTGATCCTTGGAAGTCTCCTGAGAATATTTCTCATTCT
GAGCAGCTGAAGGAGAAGGAGAAGCAGGGGTTTTTTAGGTCTATGAAGAAGAAGAAGAAGAAGT
CTCAGACAGTGCCTAATTCTGATTCTCCTGATCTGCTGACACTGCAGAAGTCTATTCATTCTGC
TTCTACACCTTCTTCTAGGCCTAAGGAGTGGAGGCCTGAGAAGATTTCTGATCTGCAGACACAG
TCTCAGCCTCTGAAGTCTCTGAGGAAGCTGCTGCATCTGTCTTCTGCTTCTAATCATCCTGCTT
CTTCTGATCCTAGGTTTCAGCCTCTGACAGCTCAGCAGACAAAGAATTCTTTTTCTGAGATTAG
GATTCATCCTCTGTCTCAGGCTTCTGGGGGGTCTTCTAATATTAGGCAGGAGCCTGCTCCTAAG
GGGAGGCCTGCTCTGCAGCTGCCTGGGCAGATGGATCCTGGGTGGCATGTGTCTTCTGTGACAA
GGTCTGCTACAGAGGGGCCTTCTTATTCTGAGCAGCTGGGGGCTAAGTCTGGGCCTAATGGGCA
TCCTTATAATAGGACAAATAGGTCTAGGATGCCTAATCTGAATGATCTGAAGGAGACAGCTCTG
SEQ ID NO: 15 (CDKL5 Brain Isoform-Isoform 2-codon-opt 2)
ATGAAGATTCCAAATATTGGGAATGTGATGAATAAGTTTGAGATTCTGGGGGTGGTGGGGGAGG
GGGCATATGGGGTGGTGCTGAAGTGTAGGCATAAGGAGACACATGAGATTGTGGCAATTAAGAA
GTTTAAGGATTCAGAGGAGAATGAGGAGGTGAAGGAGACAACACTGAGGGAGCTGAAGATGCTG
AGGACACTGAAGCAGGAGAATATTGTGGAGCTGAAGGAGGCATTTAGGAGGAGGGGGAAGCTGT
ATCTGGTGTTTGAGTATGTGGAGAAGAATATGCTGGAGCTGCTGGAGGAGATGCCAAATGGGGT
GCCACCAGAGAAGGTGAAGTCATATATTTATCAGCTGATTAAGGCAATTCATTGGTGTCATAAG
AATGATATTGTGCATAGGGATATTAAGCCAGAGAATCTGCTGATTTCACATAATGATGTGCTGA
AGCTGTGTGATTTTGGGTTTGCAAGGAATCTGTCAGAGGGGAATAATGCAAATTATACAGAGTA
TGTGGCAACAAGGTGGTATAGGTCACCAGAGCTGCTGCTGGGGGCACCATATGGGAAGTCAGTG
GATATGTGGTCAGTGGGGTGTATTCTGGGGGAGCTGTCAGATGGGCAGCCACTGTTTCCAGGGG
AGTCAGAGATTGATCAGCTGTTTACAATTCAGAAGGTGCTGGGGCCACTGCCATCAGAGCAGAT
GAAGCTGTTTTATTCAAATCCAAGGTTTCATGGGCTGAGGTTTCCAGCAGTGAATCATCCACAG
TCACTGGAGAGGAGGTATCTGGGGATTCTGAATTCAGTGCTGCTGGATCTGATGAAGAATCTGC
TGAAGCTGGATCCAGCAGATAGGTATCTGACAGAGCAGTGTCTGAATCATCCAACATTTCAGAC
ACAGAGGCTGCTGGATAGGTCACCATCAAGGTCAGCAAAGAGGAAGCCATATCATGTGGAGTCA
TCAACACTGTCAAATAGGAATCAGGCAGGGAAGTCAACAGCACTGCAGTCACATCATAGGTCAA
ATTCAAAGGATATTCAGAATCTGTCAGTGGGGCTGCCAAGGGCAGATGAGGGGCTGCCAGCAAA
TGAGTCATTTCTGAATGGGAATCTGGCAGGGGCATCACTGTCACCACTGCATACAAAGACATAT
CAGGCATCATCACAGCCAGGGTCAACATCAAAGGATCTGACAAATAATAATATTCCACATCTGC
TGTCACCAAAGGAGGCAAAGTCAAAGACAGAGTTTGATTTTAATATTGATCCAAAGCCATCAGA
GGGGCCAGGGACAAAGTATCTGAAGTCAAATTCAAGGTCACAGCAGAATAGGCATTCATTTATG
GAGTCATCACAGTCAAAGGCAGGGACACTGCAGCCAAATGAGAAGCAGTCAAGGCATTCATATA
TTGATACAATTCCACAGTCATCAAGGTCACCATCATATAGGACAAAGGCAAAGTCACATGGGGC
ACTGTCAGATTCAAAGTCAGTGTCAAATCTGTCAGAGGCAAGGGCACAGATTGCAGAGCCATCA
ACATCAAGGTATTTTCCATCATCATGTCTGGATCTGAATTCACCAACATCACCAACACCAACAA
GGCATTCAGATACAAGGACACTGCTGTCACCATCAGGGAGGAATAATAGGAATGAGGGGACACT
GGATTCAAGGAGGACAACAACAAGGCATTCAAAGACAATGGAGGAGCTGAAGCTGCCAGAGCAT
ATGGATTCATCACATTCACATTCACTGTCAGCACCACATGAGTCATTTTCATATGGGCTGGGGT
ATACATCACCATTTTCATCACAGCAGAGGCCACATAGGCATTCAATGTATGTGACAAGGGATAA
GGTGAGGGCAAAGGGGCTGGATGGGTCACTGTCAATTGGGCAGGGGATGGCAGCAAGGGCAAAT
TCACTGCAGCTGCTGTCACCACAGCCAGGGGAGCAGCTGCCACCAGAGATGACAGTGGCAAGGT
CATCAGTGAAGGAGACATCAAGGGAGGGGACATCATCATTTCATACAAGGCAGAAGTCAGAGGG
GGGGGTGTATCATGATCCACATTCAGATGATGGGACAGCACCAAAGGAGAATAGGCATCTGTAT
AATGATCCAGTGCCAAGGAGGGTGGGGTCATTTTATAGGGTGCCATCACCAAGGCCAGATAATT
CATTTCATGAGAATAATGTGTCAACAAGGGTGTCATCACTGCCATCAGAGTCATCATCAGGGAC
AAATCATTCAAAGAGGCAGCCAGCATTTGATCCATGGAAGTCACCAGAGAATATTTCACATTCA
GAGCAGCTGAAGGAGAAGGAGAAGCAGGGGTTTTTTAGGTCAATGAAGAAGAAGAAGAAGAAGT
CACAGACAGTGCCAAATTCAGATTCACCAGATCTGCTGACACTGCAGAAGTCAATTCATTCAGC
ATCAACACCATCATCAAGGCCAAAGGAGTGGAGGCCAGAGAAGATTTCAGATCTGCAGACACAG
TCACAGCCACTGAAGTCACTGAGGAAGCTGCTGCATCTGTCATCAGCATCAAATCATCCAGCAT
CATCAGATCCAAGGTTTCAGCCACTGACAGCACAGCAGACAAAGAATTCATTTTCAGAGATTAG
GATTCATCCACTGTCACAGGCATCAGGGGGGTCATCAAATATTAGGCAGGAGCCAGCACCAAAG
GGGAGGCCAGCACTGCAGCTGCCAGGGCAGATGGATCCAGGGTGGCATGTGTCATCAGTGACAA
GGTCAGCAACAGAGGGGCCATCATATTCAGAGCAGCTGGGGGCAAAGTCAGGGCCAAATGGGCA
TCCATATAATAGGACAAATAGGTCAAGGATGCCAAATCTGAATGATCTGAAGGAGACAGCACTG
SEQ ID NO: 16 (CDKL5 Brain Isoform-Isoform 2-codon-opt 3)
ATGAAGATACCAAATATAGGTAATGTAATGAATAAGTTTGAAATACTAGGTGTAGTAGGTGAAG
GTGCATATGGTGTAGTACTAAAGTGTAGGCATAAGGAAACACATGAAATAGTAGCAATAAAGAA
GTTTAAGGATTCAGAAGAAAATGAAGAAGTAAAGGAAACAACACTAAGGGAACTAAAGATGCTA
AGGACACTAAAGCAAGAAAATATAGTAGAACTAAAGGAAGCATTTAGGAGGAGGGGTAAGCTAT
ATCTAGTATTTGAATATGTAGAAAAGAATATGCTAGAACTACTAGAAGAAATGCCAAATGGTGT
ACCACCAGAAAAGGTAAAGTCATATATATATCAACTAATAAAGGCAATACATTGGTGTCATAAG
AATGATATAGTACATAGGGATATAAAGCCAGAAAATCTACTAATATCACATAATGATGTACTAA
AGCTATGTGATTTTGGTTTTGCAAGGAATCTATCAGAAGGTAATAATGCAAATTATACAGAATA
TGTAGCAACAAGGTGGTATAGGTCACCAGAACTACTACTAGGTGCACCATATGGTAAGTCAGTA
GATATGTGGTCAGTAGGTTGTATACTAGGTGAACTATCAGATGGTCAACCACTATTTCCAGGTG
AATCAGAAATAGATCAACTATTTACAATACAAAAGGTACTAGGTCCACTACCATCAGAACAAAT
GAAGCTATTTTATTCAAATCCAAGGTTTCATGGTCTAAGGTTTCCAGCAGTAAATCATCCACAA
TCACTAGAAAGGAGGTATCTAGGTATACTAAATTCAGTACTACTAGATCTAATGAAGAATCTAC
TAAAGCTAGATCCAGCAGATAGGTATCTAACAGAACAATGTCTAAATCATCCAACATTTCAAAC
ACAAAGGCTACTAGATAGGTCACCATCAAGGTCAGCAAAGAGGAAGCCATATCATGTAGAATCA
TCAACACTATCAAATAGGAATCAAGCAGGTAAGTCAACAGCACTACAATCACATCATAGGTCAA
ATTCAAAGGATATACAAAATCTATCAGTAGGTCTACCAAGGGCAGATGAAGGTCTACCAGCAAA
TGAATCATTTCTAAATGGTAATCTAGCAGGTGCATCACTATCACCACTACATACAAAGACATAT
CAAGCATCATCACAACCAGGTTCAACATCAAAGGATCTAACAAATAATAATATACCACATCTAC
TATCACCAAAGGAAGCAAAGTCAAAGACAGAATTTGATTTTAATATAGATCCAAAGCCATCAGA
AGGTCCAGGTACAAAGTATCTAAAGTCAAATTCAAGGTCACAACAAAATAGGCATTCATTTATG
GAATCATCACAATCAAAGGCAGGTACACTACAACCAAATGAAAAGCAATCAAGGCATTCATATA
TAGATACAATACCACAATCATCAAGGTCACCATCATATAGGACAAAGGCAAAGTCACATGGTGC
ACTATCAGATTCAAAGTCAGTATCAAATCTATCAGAAGCAAGGGCACAAATAGCAGAACCATCA
ACATCAAGGTATTTTCCATCATCATGTCTAGATCTAAATTCACCAACATCACCAACACCAACAA
GGCATTCAGATACAAGGACACTACTATCACCATCAGGTAGGAATAATAGGAATGAAGGTACACT
AGATTCAAGGAGGACAACAACAAGGCATTCAAAGACAATGGAAGAACTAAAGCTACCAGAACAT
ATGGATTCATCACATTCACATTCACTATCAGCACCACATGAATCATTTTCATATGGTCTAGGTT
ATACATCACCATTTTCATCACAACAAAGGCCACATAGGCATTCAATGTATGTAACAAGGGATAA
GGTAAGGGCAAAGGGTCTAGATGGTTCACTATCAATAGGTCAAGGTATGGCAGCAAGGGCAAAT
TCACTACAACTACTATCACCACAACCAGGTGAACAACTACCACCAGAAATGACAGTAGCAAGGT
CATCAGTAAAGGAAACATCAAGGGAAGGTACATCATCATTTCATACAAGGCAAAAGTCAGAAGG
TGGTGTATATCATGATCCACATTCAGATGATGGTACAGCACCAAAGGAAAATAGGCATCTATAT
AATGATCCAGTACCAAGGAGGGTAGGTTCATTTTATAGGGTACCATCACCAAGGCCAGATAATT
CATTTCATGAAAATAATGTATCAACAAGGGTATCATCACTACCATCAGAATCATCATCAGGTAC
AAATCATTCAAAGAGGCAACCAGCATTTGATCCATGGAAGTCACCAGAAAATATATCACATTCA
GAACAACTAAAGGAAAAGGAAAAGCAAGGTTTTTTTAGGTCAATGAAGAAGAAGAAGAAGAAGT
CACAAACAGTACCAAATTCAGATTCACCAGATCTACTAACACTACAAAAGTCAATACATTCAGC
ATCAACACCATCATCAAGGCCAAAGGAATGGAGGCCAGAAAAGATATCAGATCTACAAACACAA
TCACAACCACTAAAGTCACTAAGGAAGCTACTACATCTATCATCAGCATCAAATCATCCAGCAT
CATCAGATCCAAGGTTTCAACCACTAACAGCACAACAAACAAAGAATTCATTTTCAGAAATAAG
GATACATCCACTATCACAAGCATCAGGTGGTTCATCAAATATAAGGCAAGAACCAGCACCAAAG
GGTAGGCCAGCACTACAACTACCAGGTCAAATGGATCCAGGTTGGCATGTATCATCAGTAACAA
GGTCAGCAACAGAAGGTCCATCATATTCAGAACAACTAGGTGCAAAGTCAGGTCCAAATGGTCA
TCCATATAATAGGACAAATAGGTCAAGGATGCCAAATCTAAATGATCTAAAGGAAACAGCACTA
SEQ ID NO: 17 (CDKL5 Canonical Isoform-Isoform 1-codon-opt 1)
ATGAAGATTCCTAATATTGGGAATGTGATGAATAAGTTTGAGATTCTGGGGGTGGTGGGGGAGG
GGGCTTATGGGGTGGTGCTGAAGTGTAGGCATAAGGAGACACATGAGATTGTGGCTATTAAGAA
GTTTAAGGATTCTGAGGAGAATGAGGAGGTGAAGGAGACAACACTGAGGGAGCTGAAGATGCTG
AGGACACTGAAGCAGGAGAATATTGTGGAGCTGAAGGAGGCTTTTAGGAGGAGGGGGAAGCTGT
ATCTGGTGTTTGAGTATGTGGAGAAGAATATGCTGGAGCTGCTGGAGGAGATGCCTAATGGGGT
GCCTCCTGAGAAGGTGAAGTCTTATATTTATCAGCTGATTAAGGCTATTCATTGGTGTCATAAG
AATGATATTGTGCATAGGGATATTAAGCCTGAGAATCTGCTGATTTCTCATAATGATGTGCTGA
AGCTGTGTGATTTTGGGTTTGCTAGGAATCTGTCTGAGGGGAATAATGCTAATTATACAGAGTA
TGTGGCTACAAGGTGGTATAGGTCTCCTGAGCTGCTGCTGGGGGCTCCTTATGGGAAGTCTGTG
GATATGTGGTCTGTGGGGTGTATTCTGGGGGAGCTGTCTGATGGGCAGCCTCTGTTTCCTGGGG
AGTCTGAGATTGATCAGCTGTTTACAATTCAGAAGGTGCTGGGGCCTCTGCCTTCTGAGCAGAT
GAAGCTGTTTTATTCTAATCCTAGGTTTCATGGGCTGAGGTTTCCTGCTGTGAATCATCCTCAG
TCTCTGGAGAGGAGGTATCTGGGGATTCTGAATTCTGTGCTGCTGGATCTGATGAAGAATCTGC
TGAAGCTGGATCCTGCTGATAGGTATCTGACAGAGCAGTGTCTGAATCATCCTACATTTCAGAC
ACAGAGGCTGCTGGATAGGTCTCCTTCTAGGTCTGCTAAGAGGAAGCCTTATCATGTGGAGTCT
TCTACACTGTCTAATAGGAATCAGGCTGGGAAGTCTACAGCTCTGCAGTCTCATCATAGGTCTA
ATTCTAAGGATATTCAGAATCTGTCTGTGGGGCTGCCTAGGGCTGATGAGGGGCTGCCTGCTAA
TGAGTCTTTTCTGAATGGGAATCTGGCTGGGGCTTCTCTGTCTCCTCTGCATACAAAGACATAT
CAGGCTTCTTCTCAGCCTGGGTCTACATCTAAGGATCTGACAAATAATAATATTCCTCATCTGC
TGTCTCCTAAGGAGGCTAAGTCTAAGACAGAGTTTGATTTTAATATTGATCCTAAGCCTTCTGA
GGGGCCTGGGACAAAGTATCTGAAGTCTAATTCTAGGTCTCAGCAGAATAGGCATTCTTTTATG
GAGTCTTCTCAGTCTAAGGCTGGGACACTGCAGCCTAATGAGAAGCAGTCTAGGCATTCTTATA
TTGATACAATTCCTCAGTCTTCTAGGTCTCCTTCTTATAGGACAAAGGCTAAGTCTCATGGGGC
TCTGTCTGATTCTAAGTCTGTGTCTAATCTGTCTGAGGCTAGGGCTCAGATTGCTGAGCCTTCT
ACATCTAGGTATTTTCCTTCTTCTTGTCTGGATCTGAATTCTCCTACATCTCCTACACCTACAA
GGCATTCTGATACAAGGACACTGCTGTCTCCTTCTGGGAGGAATAATAGGAATGAGGGGACACT
GGATTCTAGGAGGACAACAACAAGGCATTCTAAGACAATGGAGGAGCTGAAGCTGCCTGAGCAT
ATGGATTCTTCTCATTCTCATTCTCTGTCTGCTCCTCATGAGTCTTTTTCTTATGGGCTGGGGT
ATACATCTCCTTTTTCTTCTCAGCAGAGGCCTCATAGGCATTCTATGTATGTGACAAGGGATAA
GGTGAGGGCTAAGGGGCTGGATGGGTCTCTGTCTATTGGGCAGGGGATGGCTGCTAGGGCTAAT
TCTCTGCAGCTGCTGTCTCCTCAGCCTGGGGAGCAGCTGCCTCCTGAGATGACAGTGGCTAGGT
CTTCTGTGAAGGAGACATCTAGGGAGGGGACATCTTCTTTTCATACAAGGCAGAAGTCTGAGGG
GGGGGTGTATCATGATCCTCATTCTGATGATGGGACAGCTCCTAAGGAGAATAGGCATCTGTAT
AATGATCCTGTGCCTAGGAGGGTGGGGTCTTTTTATAGGGTGCCTTCTCCTAGGCCTGATAATT
CTTTTCATGAGAATAATGTGTCTACAAGGGTGTCTTCTCTGCCTTCTGAGTCTTCTTCTGGGAC
AAATCATTCTAAGAGGCAGCCTGCTTTTGATCCTTGGAAGTCTCCTGAGAATATTTCTCATTCT
GAGCAGCTGAAGGAGAAGGAGAAGCAGGGGTTTTTTAGGTCTATGAAGAAGAAGAAGAAGAAGT
CTCAGACAGTGCCTAATTCTGATTCTCCTGATCTGCTGACACTGCAGAAGTCTATTCATTCTGC
TTCTACACCTTCTTCTAGGCCTAAGGAGTGGAGGCCTGAGAAGATTTCTGATCTGCAGACACAG
TCTCAGCCTCTGAAGTCTCTGAGGAAGCTGCTGCATCTGTCTTCTGCTTCTAATCATCCTGCTT
CTTCTGATCCTAGGTTTCAGCCTCTGACAGCTCAGCAGACAAAGAATTCTTTTTCTGAGATTAG
GATTCATCCTCTGTCTCAGGCTTCTGGGGGGTCTTCTAATATTAGGCAGGAGCCTGCTCCTAAG
GGGAGGCCTGCTCTGCAGCTGCCTGATGGGGGGTGTGATGGGAGGAGGCAGAGGCATCATTCTG
GGCCTCAGGATAGGAGGTTTATGCTGAGGACAACAGAGCAGCAGGGGGAGTATTTTTGTTGTGG
GGATCCTAAGAAGCCTCATACACCTTGTGTGCCTAATAGGGCTCTGCATAGGCCTATTTCTTCT
CCTGCTCCTTATCCTGTGCTGCAGGTGAGGGGGACATCTATGTGTCCTACACTGCAGGTGAGGG
GGACAGATGCTTTTTCTTGTCCTACACAGCAGTCTGGGTTTTCTTTTTTTGTGAGGCATGTGAT
GAGGGAGGCTCTGATTCATAGGGCTCAGGTGAATCAGGCTGCTCTGCTGACATATCATGAGAAT
GCTGCTCTGACAGGGAAG
SEQ ID NO: 18 (CDKL5 Canonical Isoform-Isoform 1-codon-opt 2)
ATGAAGATTCCAAATATTGGGAATGTGATGAATAAGTTTGAGATTCTGGGGGTGGTGGGGGAGG
GGGCATATGGGGTGGTGCTGAAGTGTAGGCATAAGGAGACACATGAGATTGTGGCAATTAAGAA
GTTTAAGGATTCAGAGGAGAATGAGGAGGTGAAGGAGACAACACTGAGGGAGCTGAAGATGCTG
AGGACACTGAAGCAGGAGAATATTGTGGAGCTGAAGGAGGCATTTAGGAGGAGGGGGAAGCTGT
ATCTGGTGTTTGAGTATGTGGAGAAGAATATGCTGGAGCTGCTGGAGGAGATGCCAAATGGGGT
GCCACCAGAGAAGGTGAAGTCATATATTTATCAGCTGATTAAGGCAATTCATTGGTGTCATAAG
AATGATATTGTGCATAGGGATATTAAGCCAGAGAATCTGCTGATTTCACATAATGATGTGCTGA
AGCTGTGTGATTTTGGGTTTGCAAGGAATCTGTCAGAGGGGAATAATGCAAATTATACAGAGTA
TGTGGCAACAAGGTGGTATAGGTCACCAGAGCTGCTGCTGGGGGCACCATATGGGAAGTCAGTG
GATATGTGGTCAGTGGGGTGTATTCTGGGGGAGCTGTCAGATGGGCAGCCACTGTTTCCAGGGG
AGTCAGAGATTGATCAGCTGTTTACAATTCAGAAGGTGCTGGGGCCACTGCCATCAGAGCAGAT
GAAGCTGTTTTATTCAAATCCAAGGTTTCATGGGCTGAGGTTTCCAGCAGTGAATCATCCACAG
TCACTGGAGAGGAGGTATCTGGGGATTCTGAATTCAGTGCTGCTGGATCTGATGAAGAATCTGC
TGAAGCTGGATCCAGCAGATAGGTATCTGACAGAGCAGTGTCTGAATCATCCAACATTTCAGAC
ACAGAGGCTGCTGGATAGGTCACCATCAAGGTCAGCAAAGAGGAAGCCATATCATGTGGAGTCA
TCAACACTGTCAAATAGGAATCAGGCAGGGAAGTCAACAGCACTGCAGTCACATCATAGGTCAA
ATTCAAAGGATATTCAGAATCTGTCAGTGGGGCTGCCAAGGGCAGATGAGGGGCTGCCAGCAAA
TGAGTCATTTCTGAATGGGAATCTGGCAGGGGCATCACTGTCACCACTGCATACAAAGACATAT
CAGGCATCATCACAGCCAGGGTCAACATCAAAGGATCTGACAAATAATAATATTCCACATCTGC
TGTCACCAAAGGAGGCAAAGTCAAAGACAGAGTTTGATTTTAATATTGATCCAAAGCCATCAGA
GGGGCCAGGGACAAAGTATCTGAAGTCAAATTCAAGGTCACAGCAGAATAGGCATTCATTTATG
GAGTCATCACAGTCAAAGGCAGGGACACTGCAGCCAAATGAGAAGCAGTCAAGGCATTCATATA
TTGATACAATTCCACAGTCATCAAGGTCACCATCATATAGGACAAAGGCAAAGTCACATGGGGC
ACTGTCAGATTCAAAGTCAGTGTCAAATCTGTCAGAGGCAAGGGCACAGATTGCAGAGCCATCA
ACATCAAGGTATTTTCCATCATCATGTCTGGATCTGAATTCACCAACATCACCAACACCAACAA
GGCATTCAGATACAAGGACACTGCTGTCACCATCAGGGAGGAATAATAGGAATGAGGGGACACT
GGATTCAAGGAGGACAACAACAAGGCATTCAAAGACAATGGAGGAGCTGAAGCTGCCAGAGCAT
ATGGATTCATCACATTCACATTCACTGTCAGCACCACATGAGTCATTTTCATATGGGCTGGGGT
ATACATCACCATTTTCATCACAGCAGAGGCCACATAGGCATTCAATGTATGTGACAAGGGATAA
GGTGAGGGCAAAGGGGCTGGATGGGTCACTGTCAATTGGGCAGGGGATGGCAGCAAGGGCAAAT
TCACTGCAGCTGCTGTCACCACAGCCAGGGGAGCAGCTGCCACCAGAGATGACAGTGGCAAGGT
CATCAGTGAAGGAGACATCAAGGGAGGGGACATCATCATTTCATACAAGGCAGAAGTCAGAGGG
GGGGGTGTATCATGATCCACATTCAGATGATGGGACAGCACCAAAGGAGAATAGGCATCTGTAT
AATGATCCAGTGCCAAGGAGGGTGGGGTCATTTTATAGGGTGCCATCACCAAGGCCAGATAATT
CATTTCATGAGAATAATGTGTCAACAAGGGTGTCATCACTGCCATCAGAGTCATCATCAGGGAC
AAATCATTCAAAGAGGCAGCCAGCATTTGATCCATGGAAGTCACCAGAGAATATTTCACATTCA
GAGCAGCTGAAGGAGAAGGAGAAGCAGGGGTTTTTTAGGTCAATGAAGAAGAAGAAGAAGAAGT
CACAGACAGTGCCAAATTCAGATTCACCAGATCTGCTGACACTGCAGAAGTCAATTCATTCAGC
ATCAACACCATCATCAAGGCCAAAGGAGTGGAGGCCAGAGAAGATTTCAGATCTGCAGACACAG
TCACAGCCACTGAAGTCACTGAGGAAGCTGCTGCATCTGTCATCAGCATCAAATCATCCAGCAT
CATCAGATCCAAGGTTTCAGCCACTGACAGCACAGCAGACAAAGAATTCATTTTCAGAGATTAG
GATTCATCCACTGTCACAGGCATCAGGGGGGTCATCAAATATTAGGCAGGAGCCAGCACCAAAG
GGGAGGCCAGCACTGCAGCTGCCAGATGGGGGGTGTGATGGGAGGAGGCAGAGGCATCATTCAG
GGCCACAGGATAGGAGGTTTATGCTGAGGACAACAGAGCAGCAGGGGGAGTATTTTTGTTGTGG
GGATCCAAAGAAGCCACATACACCATGTGTGCCAAATAGGGCACTGCATAGGCCAATTTCATCA
CCAGCACCATATCCAGTGCTGCAGGTGAGGGGGACATCAATGTGTCCAACACTGCAGGTGAGGG
GGACAGATGCATTTTCATGTCCAACACAGCAGTCAGGGTTTTCATTTTTTGTGAGGCATGTGAT
GAGGGAGGCACTGATTCATAGGGCACAGGTGAATCAGGCAGCACTGCTGACATATCATGAGAAT
GCAGCACTGACAGGGAAG
SEQ ID NO: 19 (CDKL5 Canonical Isoform-Isoform 1-codon-opt 3)
ATGAAGATACCAAATATAGGTAATGTAATGAATAAGTTTGAAATACTAGGTGTAGTAGGTGAAG
GTGCATATGGTGTAGTACTAAAGTGTAGGCATAAGGAAACACATGAAATAGTAGCAATAAAGAA
GTTTAAGGATTCAGAAGAAAATGAAGAAGTAAAGGAAACAACACTAAGGGAACTAAAGATGCTA
AGGACACTAAAGCAAGAAAATATAGTAGAACTAAAGGAAGCATTTAGGAGGAGGGGTAAGCTAT
ATCTAGTATTTGAATATGTAGAAAAGAATATGCTAGAACTACTAGAAGAAATGCCAAATGGTGT
ACCACCAGAAAAGGTAAAGTCATATATATATCAACTAATAAAGGCAATACATTGGTGTCATAAG
AATGATATAGTACATAGGGATATAAAGCCAGAAAATCTACTAATATCACATAATGATGTACTAA
AGCTATGTGATTTTGGTTTTGCAAGGAATCTATCAGAAGGTAATAATGCAAATTATACAGAATA
TGTAGCAACAAGGTGGTATAGGTCACCAGAACTACTACTAGGTGCACCATATGGTAAGTCAGTA
GATATGTGGTCAGTAGGTTGTATACTAGGTGAACTATCAGATGGTCAACCACTATTTCCAGGTG
AATCAGAAATAGATCAACTATTTACAATACAAAAGGTACTAGGTCCACTACCATCAGAACAAAT
GAAGCTATTTTATTCAAATCCAAGGTTTCATGGTCTAAGGTTTCCAGCAGTAAATCATCCACAA
TCACTAGAAAGGAGGTATCTAGGTATACTAAATTCAGTACTACTAGATCTAATGAAGAATCTAC
TAAAGCTAGATCCAGCAGATAGGTATCTAACAGAACAATGTCTAAATCATCCAACATTTCAAAC
ACAAAGGCTACTAGATAGGTCACCATCAAGGTCAGCAAAGAGGAAGCCATATCATGTAGAATCA
TCAACACTATCAAATAGGAATCAAGCAGGTAAGTCAACAGCACTACAATCACATCATAGGTCAA
ATTCAAAGGATATACAAAATCTATCAGTAGGTCTACCAAGGGCAGATGAAGGTCTACCAGCAAA
TGAATCATTTCTAAATGGTAATCTAGCAGGTGCATCACTATCACCACTACATACAAAGACATAT
CAAGCATCATCACAACCAGGTTCAACATCAAAGGATCTAACAAATAATAATATACCACATCTAC
TATCACCAAAGGAAGCAAAGTCAAAGACAGAATTTGATTTTAATATAGATCCAAAGCCATCAGA
AGGTCCAGGTACAAAGTATCTAAAGTCAAATTCAAGGTCACAACAAAATAGGCATTCATTTATG
GAATCATCACAATCAAAGGCAGGTACACTACAACCAAATGAAAAGCAATCAAGGCATTCATATA
TAGATACAATACCACAATCATCAAGGTCACCATCATATAGGACAAAGGCAAAGTCACATGGTGC
ACTATCAGATTCAAAGTCAGTATCAAATCTATCAGAAGCAAGGGCACAAATAGCAGAACCATCA
ACATCAAGGTATTTTCCATCATCATGTCTAGATCTAAATTCACCAACATCACCAACACCAACAA
GGCATTCAGATACAAGGACACTACTATCACCATCAGGTAGGAATAATAGGAATGAAGGTACACT
AGATTCAAGGAGGACAACAACAAGGCATTCAAAGACAATGGAAGAACTAAAGCTACCAGAACAT
ATGGATTCATCACATTCACATTCACTATCAGCACCACATGAATCATTTTCATATGGTCTAGGTT
ATACATCACCATTTTCATCACAACAAAGGCCACATAGGCATTCAATGTATGTAACAAGGGATAA
GGTAAGGGCAAAGGGTCTAGATGGTTCACTATCAATAGGTCAAGGTATGGCAGCAAGGGCAAAT
TCACTACAACTACTATCACCACAACCAGGTGAACAACTACCACCAGAAATGACAGTAGCAAGGT
CATCAGTAAAGGAAACATCAAGGGAAGGTACATCATCATTTCATACAAGGCAAAAGTCAGAAGG
TGGTGTATATCATGATCCACATTCAGATGATGGTACAGCACCAAAGGAAAATAGGCATCTATAT
AATGATCCAGTACCAAGGAGGGTAGGTTCATTTTATAGGGTACCATCACCAAGGCCAGATAATT
CATTTCATGAAAATAATGTATCAACAAGGGTATCATCACTACCATCAGAATCATCATCAGGTAC
AAATCATTCAAAGAGGCAACCAGCATTTGATCCATGGAAGTCACCAGAAAATATATCACATTCA
GAACAACTAAAGGAAAAGGAAAAGCAAGGTTTTTTTAGGTCAATGAAGAAGAAGAAGAAGAAGT
CACAAACAGTACCAAATTCAGATTCACCAGATCTACTAACACTACAAAAGTCAATACATTCAGC
ATCAACACCATCATCAAGGCCAAAGGAATGGAGGCCAGAAAAGATATCAGATCTACAAACACAA
TCACAACCACTAAAGTCACTAAGGAAGCTACTACATCTATCATCAGCATCAAATCATCCAGCAT
CATCAGATCCAAGGTTTCAACCACTAACAGCACAACAAACAAAGAATTCATTTTCAGAAATAAG
GATACATCCACTATCACAAGCATCAGGTGGTTCATCAAATATAAGGCAAGAACCAGCACCAAAG
GGTAGGCCAGCACTACAACTACCAGATGGTGGTTGTGATGGTAGGAGGCAAAGGCATCATTCAG
GTCCACAAGATAGGAGGTTTATGCTAAGGACAACAGAACAACAAGGTGAATATTTTTGTTGTGG
TGATCCAAAGAAGCCACATACACCATGTGTACCAAATAGGGCACTACATAGGCCAATATCATCA
CCAGCACCATATCCAGTACTACAAGTAAGGGGTACATCAATGTGTCCAACACTACAAGTAAGGG
GTACAGATGCATTTTCATGTCCAACACAACAATCAGGTTTTTCATTTTTTGTAAGGCATGTAAT
GAGGGAAGCACTAATACATAGGGCACAAGTAAATCAAGCAGCACTACTAACATATCATGAAAAT
GCAGCACTAACAGGTAAG
SEQ ID NO: 20 (DTC350)
TTGGCCACTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGAGAGGGAGTGGCCAACTC
CATCACTAGGGGTTCCTAGTGCAAGTGGGTTTTAGGACCAGGATGAGGCGGGGTGGGGGTGCCT
ACCTGACGACCGACCCCGACCCACTGGACAAGCACCCAACCCCCATTCCCCAAATTGCGCATCC
CCTATCAGAGAGGGGGAGGGGAAACAGGATGCGGCGAGGCGCGTGCGCACTGCCAGCTTCAGCA
CCGCGGACAGTGCCTTCGCCCCCGCCTGGCGGCGCGCGCCACCGCCGCCTCAGCACTGAAGGCG
CGCTGACGTCACTCGCCGGTCCCCCGCAAACTCCCCTTCCCGGCCACCTTGGTCGCGTCCGCGC
CGCCGCCGGCCCAGCCGGACCGCACCACGCGAGGCGCGAGATAGGGGGGCACGGGCGCGACCAT
CTGCGCTGCGGCGCCGGCGACTCAGCGCTGCCTCAGTCTGCGGTGGGCAGCGGAGGAGTCGTGT
CGTGCCTGAGAGCGCAGGCCGCCACCATGAAGATTCCTAACATTGGTAATGTGATGAATAAATT
TGAGATCCTTGGGGTTGTAGGTGAAGGAGCCTATGGAGTTGTACTTAAATGCAGACACAAGGAA
ACACATGAAATTGTGGCGATCAAGAAATTCAAGGACAGTGAAGAAAATGAAGAAGTCAAAGAAA
CGACTTTACGAGAGCTTAAAATGCTTCGGACTCTCAAGCAGGAAAACATTGTGGAGTTGAAGGA
AGCATTTCGTCGGAGGGGAAAGTTGTACTTGGTGTTTGAGTATGTTGAAAAAAATATGCTCGAA
TTGCTGGAAGAAATGCCAAATGGAGTTCCACCTGAGAAAGTAAAAAGCTACATCTATCAGCTAA
TCAAGGCTATTCACTGGTGCCATAAGAATGATATTGTCCATCGAGATATAAAACCAGAAAATCT
CTTAATCAGCCACAATGATGTCCTAAAACTGTGTGACTTTGGTTTTGCTCGTAATCTGTCAGAA
GGCAATAATGCTAATTACACAGAGTACGTTGCCACCAGATGGTATCGGTCCCCAGAACTCTTAC
TTGGCGCTCCCTATGGAAAGTCCGTGGACATGTGGTCGGTGGGCTGTATTCTTGGGGAGCTTAG
CGATGGACAGCCTTTATTTCCTGGAGAAAGTGAAATTGACCAACTTTTTACTATTCAGAAGGTG
CTAGGACCACTTCCATCTGAGCAGATGAAGCTTTTCTACAGTAATCCTCGCTTCCATGGGCTCC
GGTTTCCAGCTGTTAACCATCCTCAGTCCTTGGAAAGAAGATACCTTGGAATTTTGAATAGTGT
TCTACTTGACCTAATGAAGAATTTACTGAAGTTGGACCCAGCTGACAGATACTTGACAGAACAG
TGTTTGAATCACCCTACATTTCAAACCCAGAGACTTCTGGATCGTTCTCCTTCAAGGTCAGCAA
AAAGAAAACCTTACCATGTGGAAAGCAGCACATTGTCTAATAGAAACCAAGCCGGCAAAAGTAC
TGCTTTGCAGTCTCACCACAGATCTAACAGCAAGGACATCCAGAACCTGAGTGTAGGCCTGCCC
CGGGCTGACGAAGGTCTCCCTGCCAATGAAAGCTTCCTAAATGGAAACCTTGCTGGAGCTAGTC
TTAGTCCACTGCACACCAAAACCTACCAAGCAAGCAGCCAGCCTGGGTCTACCAGCAAAGATCT
CACCAACAACAACATACCACACCTTCTTAGCCCAAAAGAAGCCAAGTCAAAAACAGAGTTTGAT
TTTAATATTGACCCAAAGCCTTCAGAAGGCCCAGGGACAAAGTACCTCAAGTCAAACAGCAGAT
CTCAGCAGAACCGCCACTCATTCATGGAAAGCTCTCAAAGCAAAGCTGGGACACTGCAGCCCAA
TGAAAAGCAGAGTCGGCATAGCTATATTGACACAATTCCCCAGTCCTCTAGGAGTCCCTCCTAC
AGGACCAAGGCCAAAAGCCATGGGGCACTGAGTGACTCCAAGTCTGTGAGCAACCTTTCTGAAG
CCAGGGCCCAAATTGCGGAGCCCAGTACCAGTAGGTACTTCCCATCTAGCTGCTTAGACTTGAA
TTCTCCCACCAGCCCAACCCCCACCAGACACAGTGACACGAGAACTTTGCTCAGCCCTTCTGGA
AGAAATAACCGAAATGAGGGAACGCTGGACTCACGTCGAACCACAACCAGACATTCTAAGACGA
TGGAGGAATTGAAGCTGCCGGAGCACATGGACAGTAGCCATTCCCATTCACTGTCTGCACCTCA
CGAATCTTTTTCTTATGGACTGGGCTACACCAGCCCCTTTTCTTCCCAGCAACGTCCTCATAGG
CATTCTATGTATGTGACCCGTGACAAAGTGAGAGCCAAGGGCTTGGATGGAAGCTTGAGCATAG
GGCAAGGGATGGCAGCTAGAGCCAACAGCCTGCAACTCTTGTCACCCCAGCCTGGAGAACAGCT
CCCTCCAGAGATGACTGTGGCAAGATCTTCGGTCAAAGAGACCTCCAGAGAAGGCACCTCTTCC
TTCCATACACGCCAGAAGTCTGAGGGTGGAGTGTATCATGACCCACACTCTGATGATGGCACAG
CCCCCAAAGAAAATAGACACCTATACAATGATCCTGTGCCAAGGAGAGTTGGTAGCTTTTACAG
AGTGCCATCTCCACGTCCAGACAATTCTTTCCATGAAAATAATGTGTCAACTAGAGTTTCTTCT
CTACCATCAGAGAGCAGTTCTGGAACCAACCACTCAAAAAGACAACCAGCATTCGATCCATGGA
AAAGTCCTGAAAATATTAGTCATTCAGAGCAACTCAAGGAAAAAGAGAAGCAAGGATTTTTCAG
GTCAATGAAAAAGAAAAAGAAGAAATCTCAAACAGTACCCAATTCCGACAGCCCTGATCTTCTG
ACGTTGCAGAAATCCATTCATTCTGCTAGCACTCCAAGCAGCAGACCAAAGGAGTGGCGCCCCG
AGAAGATCTCAGATCTGCAGACCCAAAGCCAGCCATTAAAATCACTGCGCAAGTTGTTACATCT
CTCTTCGGCCTCAAATCACCCGGCTTCCTCAGATCCCCGCTTCCAGCCCTTAACAGCTCAACAA
ACCAAAAATTCCTTCTCAGAAATTCGGATTCACCCCCTGAGCCAGGCCTCTGGCGGGAGCAGCA
ACATCCGGCAGGAACCCGCACCGAAGGGCAGGCCAGCCCTCCAGCTGCCAGGTCAGATGGATCC
TGGTTGGCATGTGTCCTCTGTGACCAGGAGTGCCACAGAGGGCCCTTCCTACTCTGAACAGCTG
GGTGCCAAAAGTGGGCCAAATGGGCACCCCTATAACAGAACAAATCGCTCACGAATGCCAAATC
TGAATGATTTAAAAGAGACAGCCTTGTAAGATCCAGACATGATAAGATACATTGATGAGTTTGG
ACAAACCACAACTAGAATGCAGTGAAAAAAATGCTTTATTTGTGAAATTTGTGATGCTATTGCT
TTATTTGTAACCATTATAAGCTGCAATAAACAAGTTAACAACAACAATTGCATTCATTTTATGT
TTCAGGTTCAGGGGGAGGTGTGGGAGGTTTTTTAGAGGAACCCCTAGTGATGGAGTTGGCCACT
CCCTCTCTGCGCGCTCGCTCGCTCACTGAGGCCGCCCGGGCAAAGCCCGGGCGTCGGGCGACCT
TTGGTCGCCCGGCCTCAGTGAGCGAGCGAGCGCGCAGAGAGGGAGTGGCCAA

What is claimed is:

1. A pharmaceutical composition comprising
a) recombinant adeno-associated virus (rAAV) particles;
b) a buffer;
c) a monovalent salt;
d) a polyhydric alcohol; and
e) a triblock copolymer surfactant.

2. The pharmaceutical composition of claim 1, having a pH between 7.0 and 7.4.

3. The pharmaceutical composition of claim 1, having a pH of about 7.2.

4. The pharmaceutical composition of any one of claims 1-3, wherein the buffer is a phosphate buffer.

5. The pharmaceutical composition of claim 4, wherein phosphate is present in the composition at a concentration of between 5 mM and 30 mM.

6. The pharmaceutical composition of claim 5, wherein phosphate is present in the composition at a concentration of about 10 mM.

7. The pharmaceutical composition of any one of claims 1-3, wherein the buffer is a Tris buffer.

8. The pharmaceutical composition of claim 7, wherein Tris is present in the composition at a concentration of between 5 mM and 30 mM.

9. The pharmaceutical composition of claim 8, wherein Tris is present in the composition at a concentration of about 10 mM.

10. The pharmaceutical composition of any one of claims 1-9, wherein the polyhydric alcohol is a sugar alcohol.

11. The pharmaceutical composition of claim 10, wherein the sugar alcohol is selected from the group consisting of erythritol, glycerol, isomalt, lactitol, maltitol, mannitol, sorbitol, and xylitol.

12. The pharmaceutical composition of claim 11, wherein the sugar alcohol is sorbitol.

13. The pharmaceutical composition of claim 12, wherein sorbitol is present in the composition at a concentration of at least 1%.

14. The pharmaceutical composition of claim 13, wherein sorbitol is present in the composition at a concentration of at least 5%.

15. The pharmaceutical composition of claim 12, wherein sorbitol is present in the composition at a concentration of between 0.5% and 20%.

16. The pharmaceutical composition of claim 15, wherein sorbitol is present in the composition at a concentration of between 5% and 10%.

17. The pharmaceutical composition of claim 12, wherein sorbitol is present in the composition at a concentration of about 5%.

18. The pharmaceutical composition of claim 12, wherein sorbitol is present in the composition at a concentration of about 10%.

19. The pharmaceutical composition of any one of claims 1-18, wherein the triblock copolymer surfactant is a copolymer of ethylene oxide (EO) and propylene oxide (PO).

20. The pharmaceutical composition of claim 19, wherein the triblock copolymer surfactant is a poloxamer.

21. The pharmaceutical composition of claim 20, wherein the poloxamer is poloxamer 188 (Pluronic® F68).

22. The pharmaceutical composition of claim 20 or 21, wherein the poloxamer is present in the composition at a concentration of between 0.0001% and about 0.001%.

23. The pharmaceutical composition of claim 20 or 21, wherein the poloxamer is present in the composition at a concentration of at least 0.0001%.

24. The pharmaceutical composition of claim 23, wherein the poloxamer is present in the composition at a concentration of at least 0.0005%.

25. The pharmaceutical composition of claim 24, wherein the poloxamer is present in the composition at a concentration of at least 0.001%.

26. The pharmaceutical composition of claim 21, wherein the poloxamer is present in the composition at a concentration of about 0.0001%.

27. The pharmaceutical composition of claim 21, wherein the poloxamer is present in the composition at a concentration of about 0.001%.

28. The pharmaceutical composition of any one of claims 1-27, wherein the polyhydric alcohol is sorbitol and wherein the triblock copolymer surfactant is a poloxamer.

29. The pharmaceutical composition of any one of claims 1-28, wherein the monovalent salt is NaCl, KCl, or a combination thereof.

30. The pharmaceutical composition of claim 29, wherein NaCl is present in the composition at a concentration of between 100 mM and 250 mM.

31. The pharmaceutical composition of claim 29, wherein NaCl is present in the composition at a concentration of about 130 mM.

32. The pharmaceutical composition of claim 29, wherein NaCl is present in the composition at a concentration of about 135 mM.

33. The pharmaceutical composition of any one of claims 1-32, comprising NaCl and KCl.

34. The pharmaceutical composition of any one of claims 1-33, wherein KCl is present in the composition at a concentration of between 0.5 mM and 5 mM.

35. The pharmaceutical composition of claim 34, wherein KCl is present in the composition at a concentration of about 2 mM.

36. The pharmaceutical composition of any one of claims 1-35, further comprising one or more divalent salts.

37. The pharmaceutical composition of claim 36, wherein the divalent salt is selected from the group consisting of MgCl₂, CaCl₂, and a combination thereof.

38. The pharmaceutical composition of claim 37, wherein MgCl₂ is present in the composition at a concentration of between 0.5 mM and 5 mM.

39. The pharmaceutical composition of claim 38, wherein MgCl₂ is present in the composition at a concentration of about 1 mM.

40. The pharmaceutical composition of any one of claims 37-39, wherein CaCl is present in the composition at a concentration of between 0.5 mM and 5 mM.

41. The pharmaceutical composition of claim 40, wherein CaCl₂ is present in the composition at a concentration of about 1 mM CaCl₂.

42. The pharmaceutical composition of any one of claims 1-41, wherein rAAV particles are present in the composition at a concentration of between 1×10¹⁰ and 2×10¹⁴ GC/mL.

43. The pharmaceutical composition of any one of claims 1-42, wherein the rAAV particles comprise an AAV capsid from AAV9, AAV1, AAV2, AAV3, AAV4, AAV5, AAV6, AAV7, AAV8, AAV10, AAV11, AAV12, AAV13, AAVrh10, AAVhu37, or a variant thereof.

44. The pharmaceutical composition of claim 43, wherein the rAAV particles comprise an AAV capsid from AAV9.

45. A pharmaceutical composition comprising:
a) recombinant adeno-associated virus (rAAV) particles;
b) phosphate at a concentration of between 5 mM and 30 mM;
c) NaCl at a concentration of between 100 mM and 250 mM;
d) sorbitol at a concentration of between 1% and 10%; and
e) poloxamer at a concentration of between 0.0001% and 0.001%,
wherein the composition has a pH between 7.0 and 7.4.

46. The pharmaceutical composition of claim 45, comprising:
a) recombinant adeno-associated virus (rAAV) particles;
b) phosphate at a concentration of about 10 mM;
c) NaCl at a concentration of about 135 mM;
d) sorbitol at a concentration of about 5%; and
e) poloxamer at a concentration of about 0.001%,
wherein the composition has a pH of about 7.2.

47. The pharmaceutical composition of claim 45, comprising:
a) recombinant adeno-associated virus (rAAV) particles;
b) phosphate at a concentration of about 10 mM;
c) NaCl at a concentration of about 135 mM;
d) sorbitol at a concentration of about 10%; and
e) poloxamer at a concentration of about 0.001%,
wherein the composition has a pH of about 7.2.

48. A pharmaceutical composition comprising:
a) recombinant adeno-associated virus (rAAV) particles;
b) Tris at a concentration of between 5 mM and 30 mM;
c) NaCl at a concentration of between 100 mM and 250 mM;
d) KCl at a concentration of between 0.5 mM and 5 mM;
e) MgCl₂ at a concentration of between 0.5 mM and 5 mM;
f) CaCl₂ at a concentration of between 0.5 mM and 5 mM;
d) sorbitol at a concentration of between 1% and 10%; and
e) poloxamer at a concentration of between 0.0001% and 0.001%,
wherein the composition has a pH between 7.0 and 7.4.

49. The pharmaceutical composition of claim 48, comprising:
a) recombinant adeno-associated virus (rAAV) particles;
b) about 10 mM Tris;
c) about 130 mM NaCl;
d) about 2 mM KCl;
e) about 1 mM MgCl₂;
f) about 1 mM CaCl₂;
d) about 5% sorbitol; and
e) about 0.001% poloxamer,
wherein the composition has a pH of about 7.2.

50. The pharmaceutical composition of any one of claims 45-49, wherein the poloxamer is poloxamer 188 (Pluronic® F68).

51. The pharmaceutical composition of any one of claims 45-50, wherein the rAAV particles comprise an AAV capsid from AAV9, AAV1, AAV2, AAV3, AAV4, AAV5, AAV6, AAV7, AAV8, AAV10, AAV11, AAV12, AAV13, AAVrh10, AAVhu37, or a variant thereof.

52. The pharmaceutical composition of claim 51, wherein the rAAV particles comprise an AAV capsid from AAV9.

53. The pharmaceutical composition of any one of claims 1-52 suitable for intrathecal administration.

54. The pharmaceutical composition of claim 53, wherein the intrathecal administration comprises intraventricular, lumbar, or intra-cisterna magna administration.

55. The pharmaceutical composition of any one of claims 1-54, wherein the rAAV particles comprise an AAV capsid and a vector genome packaged therein, wherein said vector genome comprises a partial or complete coding sequence for CDKL5, or a functional fragment or variant thereof.

56. The pharmaceutical composition of claim 55, wherein the coding sequence for CDKL5 comprises a nucleotide sequence selected from SEQ ID NOs: 12-18 and 19, or a nucleotide sequence at least 95% identical to any of SEQ ID NOs: 12-19.

57. The pharmaceutical composition of claim 55, wherein the vector genome comprises a nucleotide sequence of SEQ ID NO:20 or a sequence at least 95% identical thereto.

58. A method of delivering rAAV to the central nervous system of a subject, comprising a step of administering to the subject a pharmaceutical composition of any one of claims 1-57.

59. The method of claim 58, wherein the subject is a mammal.

60. The method of claim 58 or 59, wherein the step of administering comprises administration by intrathecal administration.

61. The method of claim 60, wherein the intrathecal administration comprises intraventricular, lumbar, or intra-cisterna magna administration.

62. The method of claim 61, wherein the intrathecal administration comprises intra-cisterna magna administration.
